(19) United States
(12) Patent Application Publication
(10) Pub. No.: US 2024/0024385 A1
(43) Pub. Date: Jan. 25, 2024

(54) PROBIOTIC STRAIN FOR REDUCING SERUM URIC ACID, COMPOSITION AND USE THEREOF

(71) Applicant: Ningbo Beiyijia Biotechnology Co., Ltd., Ningbo (CN)

(72) Inventors: Haifeng LIU, Ningbo (CN); Chunyan LI, Ningbo (CN); Yanhong LIU, Ningbo (CN); Yashan CHEN, Ningbo (CN); Fengtao ZHU, Ningbo (CN); Hong LIU, Ningbo (CN); Tingting PI, Ningbo (CN); Qingshan LI, Ningbo (CN)

(21) Appl. No.: 17/814,459

(22) Filed: Jul. 22, 2022

(51) Int. Cl. A61K 35/747; A61K 47/36; A61K 31/519; A61K 31/426; A61K 31/343; A61K 31/192; A61P 19/06

(52) U.S. Cl. CPC A61K 35/747; A61K 47/36; A61K 31/519; A61K 31/426; A61K 31/343; A61K 31/192; A61P 19/06

(57) ABSTRACT

Probiotics for the purine-degrading capability under the condition of a rich-nutrient medium are screened, and a culture environment relatively simulating the human intestinal tract is obtained, so that Lactobacillus reuteri strains KLR-1, KLR-3, KLR-4 and KLR-13 are obtained. The probiotic strain or the composition containing the strain has a significant serum uric acid-reducing effect in an animal model. The composition has a probiotic-containing composition and prebiotics. The probiotic-containing composition is one or more of Lactobacillus reuteri, Lactobacillus ferrnenturn, Bacillus coagulans, Lactobacillus rhamnosus, Lactobacillus casei and Lactobacillus plantarum. The prepared compound probiotic powder can significantly reduce the serum uric acid of gout patients and has the effect of dissolving urate crystals.

### STATEMENT REGARDING PRIOR DISCLOSURES BY THE INVENTOR(S)

The following disclosures are submitted under 35 U.S.C. 102(b)(1)(A) as prior disclosures by, or on behalf of, the inventor(s) of the present application: Chinese patent application publications CN113181365A (published on Jul. 30, 2021), CN113215012A (published on Aug. 6, 2021), and CN114164130A (published on Mar. 11, 2022).

### TECHNICAL FIELD

The present invention relates to the technical field of prevention and treatment of hyperuricemia and gout, particularly relates to a probiotic strain for reducing serum uric acid, a probiotic-containing composition and use thereof.

### BACKGROUND

Hyperuricemia is a chronic metabolic disease, with clinical manifestations of serum uric acid level above the normal range (>420 μmol/L in men, 360 μmol/L in women). In addition to gout caused by urate crystals, hyperuricemia patients may develop nephropathy, urolithiasis, arteriosclerosis, cardiovascular disorders, cerebrovascular disorders and the like. It is reported that the number of hyperuricemia patients in China has reached 170 million, of which more than 80 million are gout patients, and it is rapidly increasing at an annual growth rate of 9.7%. In China, gout has become the second largest metabolic disease after diabetes.

Gout is a crystal-related arthropathy caused by deposition of monosodium urate, which is directly related to hyperuricemia caused by disorders of purine metabolism and reduction of uric acid excretion, and belongs to the category of metabolic rheumatism. The clinical symptoms of gout are severe pain, edema, redness and swelling and inflammation at the joint site. The first attack usually involves simple joints, most commonly locate in the first metatarsophalangeal joint, and the subsequent attacks may involve multiple joints such as the insteps, ankles, heels, knees, wrists, fingers and elbows, accompanied by fever. There are 3%-14% gout patients with multiple joints involved at the first attack, and acute gout rarely involves the shoulder, hip, spine, sacral, sternoclavicular, acromioclavicular or temporomandibular joints. The pain gradually abates until it disappears, lasting for several days or weeks. Urate crystals in patients with chronic gouty arthritis and tophi are preferentially deposited on cartilages of various joints such as wrists, ankles, knees, elbows, metatarsophalangeal joints and finger joints, and often deposited on bursas, auricles, tendon sheaths, subcutaneous tissues, renal interstitium and other sites as well. In a few patients, urate crystals are deposited even on nasal cartilage, tongue, vocal cords, eyelids, aorta, heart valves and myocardium and accumulated as tophi, seriously endangering their health.

If left untreated or treated incorrectly, about 12% gout patients will develop tophi after 5 years, and about 70% gout patients will develop tophi after 20 years. Statistically, the cause of 90% gout patients is reduced renal uric acid excretion, which results in kidney diseases such as acute nephritis, chronic renal insufficiency and kidney stone; in addition, 50%-70% gout patients have obesity, and the proportion of patients with gout complicated with hypertension, hyperlipidemia and diabetes is 47.2%-77.7%, 67% and 12.2%-26.9%, respectively.

In the prior art, gout is usually diagnosed by clinical manifestations examination, laboratory examination and imaging examination. “At least one occurrence of joint swelling, pain or tenderness” is taken as a necessary condition for admission to the diagnosis process, and “detection of sodium urate crystals in the joint or synovial fluid, or the presence of tophi in the connective tissue” is taken as a sufficient condition for confirmation of the diagnosis. If the sufficient condition is not met, the score is assigned cumulatively according to the clinical symptoms, physical signs, laboratory and imaging examination results. The detection method for the urate crystals in the joint or synovial fluid or the tophi in the connective tissue mainly includes invasive synovial fluid extraction and microscopic examination and noninvasive musculoskeletal ultrasound and dual-energy CT. Among them, the “invasive synovial fluid extraction microscopic examination for urate crystals” is the current golden standard for gout diagnosis, but with the progress of technology, the sensitivity and specificity of musculoskeletal ultrasound and dual-energy CT have become very high. The sensitivity and specificity of the musculoskeletal ultrasound are 100% and 76.2%, respectively, and the main factor that interferes with the specificity is the deposition of calcium pyrophosphate dihydrate deposits at joints. For patients with a medical history of more than 2 years, the sensitivity and specificity of the dual-energy CT are 92.86% and 88.24%, respectively, and the main interference factor is the tendency to miss MSUs of <2 mm. MSUs with lower density that are not yet tophi are easily missed, and artifacts are commonly found in the nails and nail beds of the feet, thickened skin and calluses on the soles of the feet. The reason for this is that keratin and calluses have similar attenuation properties to MSUs under X-ray.

Under normal physiological conditions, the total amount of uric acid in the human body is about 1200 mg. Uric acid is excreted in two main ways, about ⅔ through the kidneys in the form of urine, and ⅓ through the intestinal tract in the form of feces. Uric acid in the human body is mainly generated by the metabolism of purine nucleic acids. There are two main sources of purine nucleic acids, one of which is the intake of purine nucleic acids derived from diet, such as animal innards, seafood and beer rich in purine nucleic acids, which can lead to the rise of serum uric acid level, and the other is the release of purine nucleic acids after cell apoptosis in the metabolism process of the body. For example, in the radiotherapy and chemotherapy processes of patients with solid tumors, tumor cell extinction will lead to the release of a large amount of nucleic acids, and the death of intestinal cells caused by intestinal inflammation (such as acute enteritis caused by rotavirus infection) will also lead to the release of nucleic acids, and those nucleic acids cause severe hyperuricemia after being metabolized into uric acids. For hyperuricemia patients who are not treated with radiotherapy and chemotherapy, excessive serum uric acid is often closely related to increased intake of food-derived purine nucleic acids. The prevention and treatment of hyperuricemia and gout through a diet with restricted purine intake has been widely consensus in the medical community and has been written into the “Practical Guidelines for the Diagnosis and Treatment of Hyperuricemia in Renal Diseases in China” as a basic treatment protocol. However, it is very difficult to strictly limit the intake of those purine components in daily life, because both very tasty plant and animal cells in the diet and the flavoring ingredients in food flavors contain purine precursors (nucleotides, nucleosides, etc.), and especially, the seafood and animal meat have a relatively high purine precursors content. The diet with strictly limited purine nucleic acid intake will seriously affect the quality of life of the patients.

At present, there are two main strategies for clinically treating hyperuricemia and gout, one of which is to inhibit the generation of uric acid, and most of the drugs in this strategy are xanthine oxidase inhibitors, e.g., allopurinol, febuxostat and the like; the other one is to increase the renal excretion of uric acids, and most of the drugs in this strategy are those acting on uric acid transporters and the like, e.g., probenecid, benzbromarone and the like. In addition, there are some analgesic and anti-inflammatory drugs, e.g., colchicine, glucocorticoid and the like. However, all of those drugs have great damage to liver and kidney, and should not be used in a high-dose or for a long time. Many patients give up using those drugs for treatment because they cannot tolerate the side effects. Moreover, the use of uricosuric drugs can reduce the level of serum uric acid, but increase the concentration of uric acid in urine, thereby increasing the risk of suffering from uric acid stone. Although there are several recombinant urate oxidase drugs for treating hyperuricemia, the drugs are all injection formulations, and there are no oral urate oxidase drugs available. As an exogenous protein, the urate oxidase for injection cannot be used for a long time due to their high immunogenicity, and high price because of such drugs have complicated production process. At present, it is mainly used for treating severe hyperuricemia caused by massive cell death in some cancer patients in the chemotherapy process.

In addition, for gout, the above drugs are all palliative treatment strategies for reducing the level of serum uric acid, and once the drug administration is discontinued, the serum uric acid will quickly rise to the level before the treatment. The reason for this is that the gout is fundamentally caused by: abnormal expression and increased activity of xanthine oxidase enriched in liver; abnormal expression of uric acid transporters in the kidney and intestinal tract; and disorders of intestinal microorganisms resulting in the enrichment of xanthine oxidase and the loss of allantoinase in the intestinal microorganisms, with metabolites inducing chronic systemic inflammation in the body, resulting in increased endogenous uric acid; however, the above drugs cannot solve the fundamental causes of gout, but only inhibit the activity of xanthine oxidase and the reabsorption capability of URAT1 to degrade serum uric acid; moreover, the dissolution of deposited urate crystals and tophi by those drugs must be performed under demanding conditions that require continuous maintenance of serum uric acid below 360 μmol/L.

As an important member of human intestinal flora, probiotics are more advantageous than drugs in the treatment of hyperuricemia. How to screen for the probiotic strains with the function of reducing serum uric acid has become a hotspot of current research. A patent (CN1812801A) of Otsuka Pharmaceutical Co., Ltd. disclosed a group of Lactobacillus fermentum strains and a Saccharomycetes strain with the capability of degrading inosine and guanosine and reducing serum uric acid. However, the strains produce uric acid when degrading inosine and guanosine, and the increased concentration of uric acid in the intestinal tract can also cause hyperuricemia after being absorbed by the intestinal tract, so that the strains are not ideal probiotic strains for reducing serum uric acid. Yang Dianbin et al of Dalian Medical University reported that they have screened for a Lactobacillus brevis strain (DM9218) with the capability of degrading inosine and guanosine.

However, there is no test to determine whether the strain has the ability to degrade nucleotides. It is well known that the majority of purine precursors in food exist in the form of DNA (deoxyribonucleic acid) and RNA (ribonucleic acid). After entering the human digestive tract, DNA and RNA are hydrolyzed into deoxynucleotides and nucleotides by pancreatic deoxyribonuclease (DNase) and ribonuclease (RNase) secreted by the intestinal tract, respectively. Further, the nucleotides are decomposed into nucleosides and phosphoric acids by nucleotidase, and the nucleosides are decomposed into bases and pentoses by nucleosidase. Food-derived purine precursors (purine nucleotides, deoxypurine nucleotides, purine nucleosides, deoxypurine nucleosides, purine bases, etc.) are generally not used for synthesizing human nucleic acids after being absorbed by the human body, but are metabolized into uric acid and excreted from the human body. The absorption efficiency of the three purine precursors is different due to their different solubility. Studies by Jun OGAWA showed that purine nucleotides and purine nucleosides are more easily absorbed by cells than purine bases (Jun OGAWA, Noda Institute for Scientific Research GRANT, 2006). According to the studies, the solubility of purine nucleotides is the highest, more than 200 times that of purine nucleosides and more than 100,000 times that of purine bases (for example, at 20° C., the solubility of guanosine monophosphate in water is 20 g/100 mL, the solubility of guanosine in water is 77.6 mg/100 mL, and the solubility of guanine in water is 0.17 mg/100 mL). Therefore, nucleotides and deoxynucleotides are the most easily absorbed by the blood and cause hyperuricemia. Efficient degradation of purine nucleotides and deoxypurine nucleotides is a crucial step to reduce the absorption of food-derived purines. However, in the disclosed screening studies of probiotics for reducing purines, nucleosides were mostly used as the screening substrates. For example, a patent (CN200480017815.5) of Otsuka Pharmaceutical Co., Ltd., Japan disclosed a group of lactic acid bacteria and yeasts for reducing purines, and in the screening process therefor, only inosine and guanosine were selected as purine substrates. A Lactobacillus gasseri strain developed by Meiji Co., Ltd., Japan (Patent No. CN102747004B) was tested for decomposition ability of purine precursor, in which inosine and guanosine were also used as substrates. This Lactobacillus strain has been developed and marketed as a yogurt product. However, recent clinical studies of this strain in hyperuricemia patients have shown that its effect of reducing serum uric acid was not significant (Hisashi Yamanaka, MODERN RHEUMATOLOGY, 2019, VOL. 29, NO. 1, 146-150), presumably related to its inability to efficiently degrade purine nucleotides. A paper published by Yang Hong et al of Shanghai Jiaotong University (Jin Fang, Yang Hong, Microbiology China, 2018, 1757-1769) and a patent (CN108486007) of Jiaxing Innocul Probiotics Co., Ltd. disclosed a Lactobacillus casei for reducing purine nucleic acids (ZM15), which has the function of degrading nucleosides (the degradation rates of adenosine and guanosine are 2.56 mmol/(h·g) and 2.57 mmol/(h·g), respectively), and has been found to have the effect of reducing serum uric acid in a hyperuricemia rat model. In addition, Zhang Yanxin et al cloned urate oxidase gene into lactic acid bacteria to construct engineered probiotics capable of reducing uric acid in the expectation of reducing the concentration of uric acid in the intestinal tract and achieving the effect of reducing serum uric acid.

However, in the analysis of the published study results of probiotics for degrading purine precursors, the inventors found that most of the studies were vague in describing the capability of degrading purine precursors (for example, the capability was expressed as a percentage of degraded nucleosides without specifying the time, or expressed as a degradation rate of sub strates/g of bacteria without specifying whether the bacterium was wet or dry weight, and the reaction conditions were different across all studies), and thus were not comparable one another. In addition, it has been found that most of the scientific studies and patents evaluated the degradation of purine precursors by probiotics by the method comprising following steps: centrifuging and collecting cultured microorganisms, adding the microorganisms into a buffer containing only purine precursor (nucleoside or nucleotide) substrates, and testing the capability of the probiotics to degrade the nucleotide or nucleoside. However, this test method has a serious drawback, that is, probiotics obtained by screening through such a reaction system containing only purine precursor substrates may not absorb and degrade the purine precursor (nucleoside, nucleotide, deoxynucleoside, deoxynucleotide, etc.) substrates under conditions of abundant nutrients in the intestinal tract after entering the human body, and thus the probiotic strains picked out may not be effective in practical applications. The reason for this is that living microorganisms need nutrients to maintain their own lives at all times, and in an environment where nutrients are deficient, the microorganisms are stressed to absorb and degrade some nutrients that are not utilized under normal conditions for survival, however, once nutrients are abundant, the microorganisms will not preferentially utilize

### SUMMARY

In order to overcome the defects in the prior art, the present invention aims to provide a probiotic strain for reducing serum uric acid, a probiotic-containing composition and use thereof. The strain and the composition can effectively reduce the concentration of serum uric acid and have the effect of dissolving urate crystals and tophi.

In order to achieve the above object, the present invention provides the following technical schemes:
The first objective of the invention is to provide a Lactobacillus reuteri strain for reducing serum uric acid, having capability of degrading purine precursors, wherein the purine precursors include, but are not limited to any one, several or all of inosine/deoxyinosine, inosine monophosphate/deoxyinosine monophosphate, guanosine/deoxyguanosine, guanosine monophosphate/deoxyguanosine monophosphate, adenosine/deoxyadenosine, and adenosine monophosphate/deoxyadenosine monophosphate.
Preferably, the Lactobacillus reuteri strain for reducing serum uric acid having an average rate of degradation of purine precursors of >50 mg/OD·h·L, >100 mg/OD·h·L, >150 mg/OD·h·L, >200 mg/OD·h·L, ≥250 mg/OD·h·L, or ≥300 mg/OD·h·L, wherein the purine precursors include at least one, several or all of inosine/deoxyinosine, inosine monophosphate/deoxyinosine monophosphate, guanosine/deoxyguanosine, guanosine monophosphate/deoxyguanosine monophosphate, adenosine/deoxyadenosine, and adenosine monophosphate/deoxyadenosine monophosphate.
Preferably, the Lactobacillus reuteri strain for reducing serum uric acid wherein the Lactobacillus reuteri strain is Lactobacillus reuteri strain KLR-1 with an accession number of CGMCC No. 18699, Lactobacillus reuteri strain KLR-3 with an accession number of CGMCC No. 18700, Lactobacillus reuteri strain KLR-13 with an accession number of CGMCC No. 19329.
Preferably, the Lactobacillus reuteri strain for reducing serum uric acid wherein the Lactobacillus reuteri strain is Lactobacillus reuteri strain KLR-4 with an accession number of CCTCC No. M2020367.
The secondary objective of the invention is to provide a probiotic-containing composition for reducing serum uric acid, comprising at least one of the Lactobacillus reuteri strains of above technical schemes.
Preferably, the probiotic-containing composition comprising the viable count of Lactobacillus reuteri strain as following:
the Lactobacillus reuteri strain KLR-1 has a viable count of 1×10⁵−5×10¹¹ CFU/g of the composition;
the Lactobacillus reuteri strain KLR-3 has a viable count of 1×10⁵−5×10¹¹ CFU/g of the composition;
the Lactobacillus reuteri strain KLR-13 has a viable count of 1×10⁵−5×10¹¹ CFU/g of the composition; and
Preferably, the probiotic-containing composition comprising the viable count of Lactobacillus reuteri strain as following:
the Lactobacillus reuteri strain KLR-1 has a viable count of 1×10⁸−5×10¹¹ CFU/g of the composition;
the Lactobacillus reuteri strain KLR-3 has a viable count of 1×10⁸−5×10¹¹ CFU/g of the composition;
the Lactobacillus reuteri strain KLR-13 has a viable count of 1×10⁸−5×10¹¹ CFU/g of the composition.
Preferably, the probiotic-containing composition comprising the Lactobacillus reuteri strain KLR-3 and the Lactobacillus reuteri strain KLR-1; the Lactobacillus reuteri strain KLR-3 and the Lactobacillus reuteri strain KLR-13.
Preferably, the probiotic-containing composition comprising the following components in parts by weight: 1-8 parts of a powder of the Lactobacillus reuteri strain KLR-1, 1-8 parts of a powder of the Lactobacillus reuteri strain KLR-3, and 1-8 parts of a powder of the Lactobacillus reuteri strain KLR-13, wherein
the Lactobacillus reuteri strain KLR-1 has a viable cell count of 1×10⁹−1×10¹² CFU/g of the powder;
the Lactobacillus reuteri strain KLR-3 has a viable cell count of 1×10⁹−1×10¹² CFU/g of the powder; and
the Lactobacillus reuteri strain KLR-3 has a viable cell count of 1×10⁹−1×10¹² CFU/g of the powder.
Meanwhile, the invention is to provide a probiotic-containing composition for reducing serum uric acid, comprising the Lactobacillus reuteri strains KLR-4 of above technical schemes.
Preferably, the probiotic-containing composition comprising the viable count of Lactobacillus reuteri KLR-4 strain is 1×10⁵−5×10¹² CFU/g of the composition.
The invention is to provide a probiotic-containing composition for reducing serum uric acid, comprising at least one of the Lactobacillus reuteri strains of above technical schemes.
Preferably, the Lactobacillus reuteri strain is Lactobacillus reuteri strain KLR-1, KLR-3, KLR-13, or KLR-4.
Preferably, the probiotic-containing composition comprising the Lactobacillus reuteri strain KLR-3 and the Lactobacillus reuteri strain KLR-1; the Lactobacillus reuteri strain KLR-3 and the Lactobacillus reuteri strain KLR-13; or the Lactobacillus reuteri strain KLR-3, the Lactobacillus reuteri strain KLR-1 and the Lactobacillus reuteri strain KLR-13; the Lactobacillus reuteri strain KLR-3 and the Lactobacillus reuteri strain KLR-4; the Lactobacillus reuteri strain KLR-3, the Lactobacillus reuteri strain KLR-1, the Lactobacillus reuteri strain KLR-13 and the Lactobacillus reuteri strain KLR-4.
Preferably,
the Lactobacillus reuteri strain KLR-1 has a viable cell count of 1×10⁵−5×10¹¹ CFU/g of the composition;
the Lactobacillus reuteri strain KLR-3 has a viable cell count of 1×10⁵−5×10¹¹ CFU/g of the composition; and
the Lactobacillus reuteri strain KLR-13 has a viable cell count of 1×10⁵−5×10¹¹ CFU/g of the composition.
the Lactobacillus reuteri strain KLR-4 has a viable cell count of 1×10⁵−1×10¹² CFU/g of the composition;
Preferably,
the Lactobacillus reuteri strain KLR-1 has a viable cell count of 1×10⁸−5×10¹⁰ CFU/g of the composition; and
the Lactobacillus reuteri strain KLR-3 has a viable cell count of 1×10⁸−5×10¹⁰ CFU/g of the composition.
the Lactobacillus reuteri strain KLR-13 has a viable cell count of 1×10⁸−1×10¹⁰ CFU/g of the composition;

the Lactobacillus reuteri strain KLR-4 has a viable cell count of 1×10⁸−1×10¹¹ CFU/g of the composition.
Preferably, the probiotic-containing composition comprising the following components in parts by weight: 1-8 parts of a powder of the Lactobacillus reuteri strain KLR-1, 1-8 parts of a powder of the Lactobacillus reuteri strain KLR-3, and 1-8 parts of a powder of the Lactobacillus reuteri strain KLR-13,1-8 parts of a powder of the Lactobacillus reuteri strain KLR-4, wherein
the Lactobacillus reuteri strain KLR-1 has a viable cell count of 1×10⁹−1×10¹² CFU/g of the powder;
the Lactobacillus reuteri strain KLR-3 has a viable cell count of 1×10⁹−1×10¹² CFU/g of the powder; and
the Lactobacillus reuteri strain KLR-13 has a viable cell count of 1×10⁹−1×10¹² CFU/g of the powder, and
the Lactobacillus reuteri strain KLR-4 has a viable cell count of 1×10⁹−1×10¹² CFU/g of the powder.
The third objective of the invention is to provide the use of the Lactobacillus reuteri strain for reducing serum uric acid described above in the preparation of a pharmaceutical or a dietary product for preventing and treating hyperuricemia and/or gout.
Preferably, the pharmaceutical is in a dosage form for oral administration, wherein the dosage form is preferably selected from: a solution, a suspension, an emulsion, a powder, a lozenge, a pill, a syrup, a troche, a tablet, a chewing gum, a concentrated syrup, and a capsule.
Preferably, the dietary product includes an ordinary food product, a health-care food product, or a food product for special medical purpose.
The fourth objective of the invention is to provide a composition capable of reducing serum uric acid and dissolving urate crystals and tophi, comprising a probiotic-containing composition and prebiotics;
wherein the probiotic-containing composition is one or more of Lactobacillus reuteri strain, Lactobacillus fermentum strain, Bacillus coagulans strain, Lactobacillus rhamnosus strain, Lactobacillus casei strain and Lactobacillus plantarum strain.

In detail, in the above composition, the strain in the probiotic-containing composition is tolerant to gastric acid and bile salt, and its metabolites can improve the activity of the Na⁺—K⁺ pump and Ca-ATPase, improve the solubility of urate, and inhibit the deposition of crystals so as to promote the dissolution of crystals, and specifically degrade purine nucleotides of food into purine nucleosides and purine bases with low solubility, which are excreted along with bacteria so as to reduce exogenous uric acid; the strain in the probiotic-containing composition improves the ecology of gut microflora and relieves the flora structure of xanthine oxidase enrichment and urate oxidase deficiency in gout patients by competitively colonizing the intestinal tract with harmful microorganisms. The prebiotics, on the one hand, assist in the growth and proliferation of the strain in the probiotic-containing composition in the intestinal tract, on the other hand, improve the gut microflora of gout patients, inhibit the growth and proliferation of xanthine oxidase strains and promote the enrichment of urate oxidase-producing strains so as to promote the intestinal excretion of uric acid.

In the above technical scheme, the composition capable of reducing uric acid and dissolving urate crystals and tophi, wherein the probiotic-containing composition and the prebiotics are added in a weight ratio of (1-80):(1-80), preferably (8-32):(40-80).

In detail, in the above technical scheme, when the composition and the prebiotics are added in a ratio within the range, the optimal survival and colonization of the probiotic-containing composition can be ensured, so that the optimal functional efficiency of the strain is achieved. Moreover, the “Jarisch-Herxheimer die-off reaction” can be avoided in gout patients.

In the above technical scheme, the probiotic-containing composition has a total viable count of 1×10⁶−6×10¹² CFU/g.

Preferably, the Lactobacillus reuteri strain, the Lactobacillus fermentum strain, the Bacillus coagulans strain, the Lactobacillus rhamnosus strain, the Lactobacillus casei strain and the Lactobacillus plantarum strain have viable counts of 1.2×10⁹−2.1×10¹² CFU/g, 1.1×10⁹−2.0×10¹² CFU/g, 1.3×10⁸−1.5×10¹¹ CFU/g, 1.3×10⁹−2.1×10¹² CFU/g, 1.0×10⁹−1.4×10¹² CFU/g and 5×10⁹−1.2×10¹² CFU/g, respectively.

In detail, in the above technical scheme, the Lactobacillus reuteri strain is one or more of Lactobacillus reuteri strain KLR-1 with an accession number of CGMCC No. 18699, the Lactobacillus reuteri strain KLR-3 with an accession number of CGMCC No. 18700, the Lactobacillus reuteri strain KLR-4 with an accession number of CCTCC No. M2020367 and the Lactobacillus reuteri strain KLR-13 with an accession number of CGMCC No. 19329;

In detail, in the above technical scheme, the Lactobacillus rhamnosus strain is Lactobacillus rhamnosus strain KLrh-10 with an accession number of CGMCC No. 19711;

In detail, in the above technical scheme, the Lactobacillus casei is Lactobacillus casei strain KLca-10 with an accession number of CGMCC No. 19708;

In detail, in the above technical scheme, the Lactobacillus plantarum is Lactobacillus plantarum strain KLp1-3 with an accession number of CCTCC No. M2020366.

In detail, in the above technical scheme, the composition capable of reducing serum uric acid and dissolving urate crystals and tophi further comprising a citrate, wherein the citrate is one or more of citric acid, citric acid esters and citric acid salts;
preferably, the probiotic-containing composition, the prebiotics and the citrate are added in a weight ratio of (1-80):(1-80):(1-50), more preferably (8-32):(40-80):(10-30);

In detail, in the above technical scheme, when the citrate is added in an amount within the range, it can not only ensure the alkalization of urine by citric acid salts so as to promote the renal excretion of uric acid, but also avoid the adverse effects caused by long-term use.

In detail, in the above technical scheme, the citrate is one or more of citric acid, potassium citrate, magnesium citrate, calcium citrate and potassium sodium hydrogen citrate.

In detail, in the above technical scheme, the prebiotics are one or more of inulin, fructo-oligosaccharide, galacto-oligosaccharide, xylo-oligosaccharide, raffinose and stachyose.

The fifth objective of the invention is to provide use of the composition in the preparation of a dietary product, a pharmaceutical and a health-care product for preventing and treating hyperuricemia and/or gout.

The sixth objective of the invention is to provide the combined use of the composition with a uric acid-reducing drug in the preparation of a food product, a pharmaceutical and a health-care product for dissolving urate crystals and tophi.

In detail, in the above technical scheme, the uric acid-reducing drug is one or more of allopurinol, febuxostat, benzbromarone and probenecid.

In detail, in the above technical scheme, the composition and the uric acid-reducing drug are added in a weight ratio of 100:1-1000:1, preferably 300:1-750:1.

### Advantages and Beneficial Effects of the Present Invention

In order to more effectively screen for the probiotics for degrading purine precursors in the simulated intestinal environment, for the first time, screening the probiotics for the purine-degrading capability under the condition of a rich-nutrient medium is proposed in the present application. By optimizing culture conditions and test methods, a method for screening for a culture environment simulating the human intestinal tract is obtained, and after multiple rounds of screening and optimization, probiotics with remarkable purine precursor-degrading capability under two test conditions of a no-nutrient system containing only purine precursors and a rich-nutrient system containing both purine precursors and nutrients of carbon and nitrogen sources are obtained, which have significantly enhanced capability as compared to a previously reported purine precursor-degrading Lactobacillus gasseri probiotic strain from Japan and a purine precursor-degrading Lactobacillus casei strain disclosed in a China patent (see Example 1 for details). By the screening in the present application, the Lactobacillus reuteri strains KLR-1 and KLR-13 picked out can degrade deoxyguanosine and deoxyadenosine in addition to inosine, guanosine and adenosine; and the Lactobacillus reuteri strain KLR-3 picked out can degrade deoxyguanosine monophosphate and deoxyadenosine monophosphate in addition to inosine monophosphate, guanosine monophosphate and adenosine monophosphate. The absorption efficiency of purine precursors is ranked as follows: nucleotides/deoxynucleotides>nucleosides/deoxynucleosides>purine bases. The uric acid-reducing effect test in an animal model shows that the above strains have a significant serum uric acid-reducing effect, and when they are at an equal dose, the Lactobacillus reuteri strain KLR-3 has a superior effect to KLR-1 and KLR-13, indicating that the efficient degradation of purine nucleotides and purine deoxynucleotides to purine bases can greatly reduce the absorbable concentration of food-derived purine precursors in the gastrointestinal tract due to the extremely low solubility of purine bases, and thus reduce the absorption of purine precursors from food. The effect of a mixture of strains is better than that of a single KLR-1/KLR-13 or KLR-3 strain, indicating that the simultaneous degradation of nucleosides/deoxynucleosides and nucleotides/deoxynucleotides can better reduce the absorption of food-derived purines.

The Lactobacillus reuteri strain KLR-4 picked out in the present invention can simultaneously and efficiently degrade guanosine and guanosine monophosphate, adenosine and adenosine monophosphate, inosine and inosine monophosphate, deoxyguanosine and deoxyguanosine monophosphate, and deoxyadenosine and deoxyadenosine monophosphate. The uric acid-reducing effect test in an animal model shows that the KLR-4 strain has a more significant uric acid-reducing effect than the KLR-1 strain only capable of degrading purine nucleosides or deoxypurine nucleosides or the KLR-3 strain only capable of degrading purine nucleotides and deoxypurine nucleotides when they are at an equal dose, and basically achieves the effect of mixed powder of KLR-1 and KLR-3 strains at an equal dose.

Lactobacillus reuteri also has distinct advantages over other reported purine precursor-degrading probiotics, that is, it has strong intestinal colonization, and the reuterin secreted by it can inhibit harmful bacteria in the intestinal tract, enhance the immunity and inhibit intestinal inflammation (Pang Jie, China Biotechnology, 2011, 31(5): 131-137; Tang Jia, Journal of Southern Medical University, 2019, 39(10): 1221-1226). The death of intestinal cells can be reduced by relieving inflammation, so that the generation of endogenous uric acid can be reduced, purine intake in diet can be synergistically degraded, and a better serum uric acid-reducing is achieved. Therefore, Lactobacillus reuteri strains capable of degrading purine precursors, as a new means for reducing serum uric acid and treating gout, significantly reduce the absorption of food-derived purines without lowering the quality of life (low-purine diet), thus achieving an effect of low-purine diet. It also has no toxic or side effect and higher safety as compared to clinical chemical treatment methods, and thus has wide application prospect. In addition, the composition capable of reducing uric acid and dissolving urate crystals and tophi is prepared by combining the probiotic-containing composition and the prebiotics, and the prepared compound probiotic powder can significantly reduce the serum uric acid of a gout patient, and the effect of the compound probiotic powder on the dissolution of urate crystals and tophi is verified by dual-energy CT for the first time.

### Notes on the Deposit of Biological Materials

Lactobacillus reuteri is deposited in the China General Microbiological Culture Collection Center (CGMCC) at No. 3, Yard No. 1, Beichen West Road, Chaoyang District, Beijing on Oct. 17, 2019, with an accession number of CGMCC No. 18699 and a strain number of KLR-1.

Lactobacillus reuteri is deposited in the China General Microbiological Culture Collection Center (CGMCC) at No. 3, Yard No. 1, Beichen West Road, Chaoyang District, Beijing on Oct. 17, 2019, with an accession number of CGMCC No. 18700 and a strain number of KLR-3.

Lactobacillus reuteri is deposited in the China Center for Type Culture Collection (CCTCC) at No. 299 Bayi Road, Wuchang District, Wuhan City, Hubei Province on Jul. 28, 2020, with an accession number of CCTCC M 2020367 and a strain number of KLR-4.

Lactobacillus reuteri is deposited in the China General Microbiological Culture Collection Center (CGMCC) at No. 3, Yard No. 1, Beichen West Road, Chaoyang District, Beijing on Jan. 10, 2020, with an accession number of CGMCC No. 19329 and a strain number of KLR-13.

Lactobacillus rhamnosus is deposited in the China General Microbiological Culture Collection Center (CGMCC) at No. 3, Yard No. 1, Beichen West Road, Chaoyang District, Beijing on Apr. 23, 2020, with an accession number of CGMCC No. 19711 and a strain number of KLrh-10.

Lactobacillus casei is deposited in the China General Microbiological Culture Collection Center (CGMCC) at No. 3, Yard No. 1, Beichen West Road, Chaoyang District, Beijing on Apr. 23, 2020, with an accession number of CGMCC No. 19708 and a strain number of KLca-10.

Lactobacillus plantarum is deposited in the China Center for Type Culture Collection (CCTCC) at No. 299 Bayi Road, Wuchang District, Wuhan City, Hubei Province on Jul. 28, 2020, with an accession number of CCTCC M 2020366 and a strain number of KLpl-3.

### BRIEF DESCRIPTION OF THE DRAWINGS

FIG. 1 Standard curves for the test concentrations of different purine precursor substrates;

FIG. 2 An LC-MS profile of the mixed standard;

FIG. 3 Dual-energy CT imaging results of a blank control group and experiment groups before and after interventions with composition 1, composition 3, composition 4 and composition 6, respectively, in the example of the present invention.

### DETAILED DESCRIPTION

The technical scheme of the present invention will be further illustrated in detail with reference to the following specific examples. It should be understood that the following examples are merely exemplary illustration and explanation of the present invention, and should not be construed as limiting the protection scope of the present invention. All techniques implemented based on the content of the present invention described above are encompassed within the protection scope of the present invention.

Unless otherwise stated, the starting materials and reagents used in the following examples are all commercially available products or can be prepared by known methods. Unless otherwise stated, the experimental methods used in the following examples are conventional methods.

### EXAMPLE 1. SCREENING OF PROBIOTICS

The probiotic strains to be tested that have been picked out and preserved in the laboratory (including 10 species of 130 Lactobacillus spp. probiotic strains such as Lactobacillus rhamnosus, Lactobacillus helveticus, Lactobacillus casei, Lactobacillus reuteri, Lactobacillus plantarum, Lactobacillus acidophilus, Lactobacillus salivarius, Lactobacillus gasseri and Lactobacillus fermentum, Bifidobacterium animalis, Bifidobacterium longum, Bifidobacterium infantis, Pediococcus acidilactici) were activated, inoculated into MRS medium, cultured at 37° C. for 8-20 h under facultative anaerobic (standing) or strictly anaerobic (oxygen concentration <0.5%) conditions, and centrifuged to collect the bacteria. The bacteria were washed with phosphate buffer (100 mM, pH 7.0) 3 times, adjusted to OD₆₀₀=2.7 (1 OD equals to about 2.0−3.0×10⁸ CFU/mL) and added to a test phosphate buffer system (20 mM, pH 6.86) containing 0.7 mg/mL adenosine monophosphate, guanosine monophosphate, inosine monophosphate, adenosine, guanosine and inosine to reach a final concentration of OD₆₀₀=0.6. The mixture was incubated at 37° C. for 1 h and centrifuged at 8000 g for 5 min to collect the supernatant. 900 μL of the supernatant was taken and added with 100 μL of 100 mM perchloric acid solution to terminate the reaction, and the mixture was filtered through a 0.22 μm filter and then detected by high performance liquid chromatograph (HPLC).

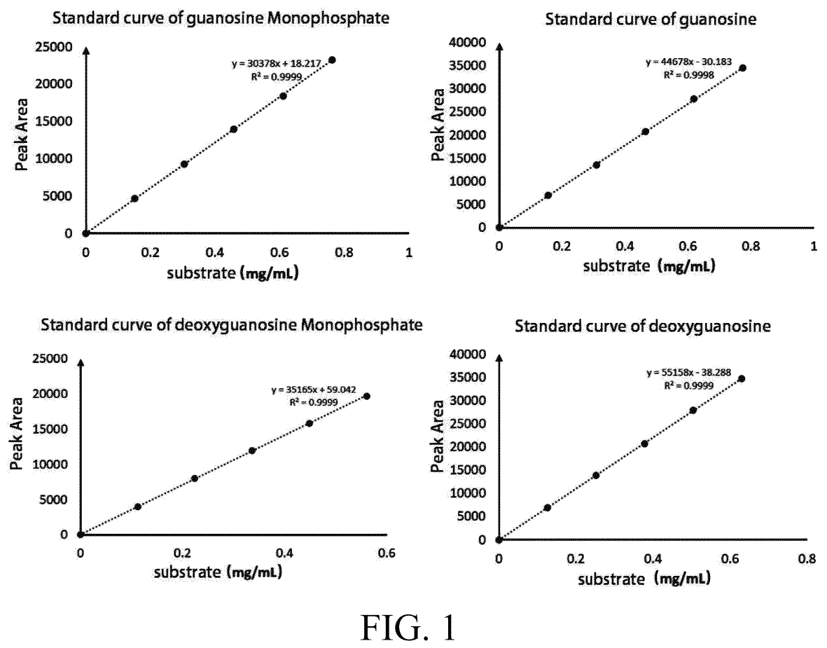

The standard curves for purine precursor substrates were obtained as follows: analytically pure (>95% purity) adenosine, deoxyadenosine, inosine, guanosine, deoxyguanosine, adenosine monophosphate, deoxyadenosine monophosphate, disodium guanosine monophosphate, deoxyguanosine monophosphate and disodium inosine monophosphate were precisely weighed, and added with sterile water to obtain standards of different gradient concentrations, followed by the above operation for terminating the reaction and HPLC detection to establish standard curves for the substrates, such as guanosine monophosphate, guanosine, deoxyguanosine monophosphate and deoxyguanosine, as shown in FIG. 1.

The specific conditions for the HPLC detection are as follows: Sepax Bio C18 column, mobile phase A: 20 mM potassium dihydrogen phosphate buffer (pH 2.5), mobile phase B: methanol, flow rate: 1.0 mL/min, detection wavelength: 254 nm, and injection volume: 20 μL.

TABLE 1
Elution gradient of HPLC

| | | | | | | |
|---|---|---|---|---|---|---|
| Time (min) | 0 | 20 | 21 | 25 | 26 | 26 |
| Mobile phase A (%) | 100 | 100 | 90 | 90 | 100 | 100 |
| Mobile phase B (%) | 0 | 0 | 10 | 10 | 0 | 0 |

TABLE 2
Retention time of the purine precursor substrates

| | | disodium | disodium | | | |
|---|---|---|---|---|---|---|
| | adenosine | inosine | guanosine | | | |
| Substrate | monophosphate | monophosphate | monophosphate | adenosine | inosine | guanosine |
| Retention | 5.16 | 5.47 | 5.78 | 10.29 | 10.54 | 13.11 |
| time(min) | | | | | | |
| Substrate | deoxyadenosine | | deoxyguanosine | deoxyadenosine | | deoxyguanosine |
| | monophosphate | | monophosphate | | | |
| Retention | 11.70 | | 6.84 | 28.56 | | 17.12 |
| time(min) | | | | | | |

The probiotic strains were screened by the purine precursor-degrading effect test according to the above detection method for the purine precursors. The results of the rate of degradation of purine precursors by the strains were determined as follows:

TABLE 3
Rate of degradation of purine precursors by different probiotic strains

| | | Rate of degradation of purine precursors (mg/OD · h · L) | | | | | |
|---|---|---|---|---|---|---|---|
| | Strain | | Guanosine | | Inosine | | Adenosine |
| Strain name | code | Guanosine | monophosphate | Inosine | monophosphate | Adenosine | monophosphate |
| Lactobacillus | KLac-1 | 35 | 27 | 8 | 6 | 14 | 12 |
| acidophilus | KLac-2 | 260 | 20 | 310 | 18 | 214 | 11 |
| | KLac-3 | 3 | 19 | 9 | 7 | 12 | 10 |
| | KLac-4 | 41 | 29 | 9 | 6 | 14 | 12 |
| | KLac-5 | 62 | 24 | 9 | 6 | 14 | 11 |
| | KLac-6 | 67 | 28 | 9 | 6 | 19 | 15 |
| | KLac-7 | 10 | 25 | 9 | 6 | 13 | 11 |
| | KLac-8 | 11 | 24 | 22 | 6 | 24 | 18 |
| | KLac-9 | 11 | 21 | 42 | 31 | 44 | 44 |
| | KLac-10 | 9 | 31 | 32 | 42 | 45 | 32 |
| | KLac-11 | 13 | 26 | 47 | 37 | 47 | 57 |
| | KLac-12 | 22 | 34 | 10 | 13 | 21 | 21 |
| | KLac-13 | 13 | 31 | 13 | 11 | 31 | 8 |
| | KLac-14 | 19 | 33 | 16 | 15 | 21 | 11 |
| | KLac-15 | 16 | 35 | 9 | 7 | 9 | 9 |
| | KLac-16 | 5 | 28 | 15 | 12 | 13 | 23 |
| | KLac-17 | 15 | 30 | 24 | 21 | 22 | 33 |
| | KLac-18 | 11 | 30 | 13 | 12 | 23 | 13 |
| | KLac-19 | 70 | 30 | 16 | 19 | 11 | 29 |
| Lactobacillus | KLca-1 | 7 | 23 | 16 | 6 | 23 | 18 |
| casei | KLca-2 | 1 | 34 | 11 | 8 | 22 | 7 |
| | KLca-3 | 2 | 18 | 11 | 8 | 21 | 7 |
| | KLca-4 | 2 | 19 | 10 | 7 | 22 | 7 |
| | KLca-5 | 3 | 17 | 10 | 7 | 22 | 28 |
| | KLca-6 | 3 | 5 | 5 | 2 | 20 | 6 |
| | KLca-7 | 3 | 38 | 16 | 3 | 16 | 3 |
| | KLca-8 | 2 | 28 | 8 | 5 | 14 | 11 |
| | KLca-9 | 122 | 39 | 176 | 43 | 213 | 29 |
| | KLca-10 | 1 | 12 | 14 | 21 | 11 | 32 |
| | KLca-11 | 2 | 39 | 5 | 22 | 18 | 22 |
| | KLca-12 | 1 | 15 | 4 | 9 | 12 | 32 |
| | ZM-15 | 134 | 147 | 141 | 111 | 126 | 97 |
| Lactobacillus | KLdB-1 | 238 | 2 | 210 | 7 | 216 | 13 |
| bulgaricus | KLdB-2 | 219 | 23 | 211 | 8 | 253 | 18 |
| | KLdB-3 | 154 | 20 | 212 | 9 | 220 | 16 |
| | KLdB-4 | 25 | 18 | 11 | 8 | 14 | 11 |
| | KLdB-5 | 7 | 13 | 8 | 5 | 14 | 11 |
| | KLdB-6 | 31 | 10 | 9 | 6 | 7 | 10 |
| | KLdB-7 | 9 | 7 | 16 | 13 | 7 | 10 |
| Lactobacillus | KLfe-1 | 253 | 39 | 210 | 7 | 302 | 5 |
| fermentium | KLfe-2 | 96 | 42 | 3 | 1 | 4 | 6 |
| | KLfe-3 | 104 | 38 | 110 | 6 | 108 | 6 |
| | KLfe-4 | 58 | 51 | 9 | 6 | 11 | 8 |
| | KLfe-5 | 456 | 20 | 510 | 6 | 409 | 7 |
| | KLfe-6 | 98 | 33 | 9 | 5 | 9 | 6 |
| | KLfe-7 | 473 | 38 | 458 | 54 | 493 | 86 |
| | KLfe-8 | 213 | 26 | 210 | 7 | 309 | 6 |
| | KLfe-9 | 10 | 7 | 10 | 7 | 9 | 6 |
| | ZM-5 | 79 | 64 | 101 | 78 | 82 | 66 |
| Lactobacillus | KLga-1 | 12 | 24 | 89 | 6 | 9 | 28 |
| gasseri | KLga-2 | 11 | 19 | 111 | 5 | 11 | 38 |
| | KLga-3 | 8 | 19 | 16 | 6 | 11 | 27 |
| | KLga-4 | 11 | 21 | 15 | 7 | 10 | 43 |
| | KLga-5 | 9 | 18 | 18 | 7 | 10 | 18 |
| | PA-3 | 10 | 7 | 13 | 2 | 5 | 2 |
| Lactobacillus | KLhe-1 | 10 | 7 | 9 | 4 | 6 | 3 |
| helveticus | KLhe-2 | 10 | 7 | 12 | 6 | 8 | 5 |
| | KLhe-3 | 12 | 8 | 12 | 7 | 10 | 7 |
| | KLhe-4 | 10 | 7 | 12 | 7 | 11 | 8 |
| | KLhe-5 | 17 | 14 | 12 | 7 | 12 | 9 |
| | KLhe-6 | 16 | 13 | 13 | 8 | 11 | 8 |
| | KLhe-7 | 15 | 12 | 6 | 8 | 6 | 14 |
| | KLhe-8 | 1 | 40 | 21 | 34 | 12 | 21 |
| | KLhe-9 | 334 | 30 | 386 | 32 | 347 | 22 |
| | KLhe-10 | 1 | 40 | 21 | 2 | 12 | 15 |
| | KLhe-11 | 2 | 35 | 11 | 8 | 13 | 24 |
| | KLhe-12 | 0 | 47 | 23 | 21 | 7 | 23 |
| Lactobacillus | KLpl-1 | 27 | 19 | 6 | 11 | 8 | 14 |
| plantarum | KLpl-2 | 46 | 13 | 6 | 9 | 7 | 12 |
| | KLpl-4 | 63 | 16 | 4 | 9 | 6 | 14 |
| | KLpl-5 | 7 | 2 | 17 | 9 | 6 | 19 |
| | KLpl-6 | 31 | 1 | 7 | 9 | 6 | 13 |
| | KLpl-7 | 57 | 5 | 43 | 15 | 27 | 45 |
| | KLpl-8 | 48 | 3 | 41 | 13 | 43 | 23 |
| | KLpl-9 | 87 | 7 | 47 | 9 | 67 | 47 |
| | KLpl-10 | 48 | 11 | 45 | 21 | 40 | 24 |
| | KLpl-11 | 83 | 6 | 63 | 36 | 72 | 26 |
| | KLpl-12 | 54 | 2 | 24 | 22 | 44 | 25 |
| | KLpl-13 | 9 | 11 | 7 | 12 | 13 | 12 |
| | KLpl-14 | 22 | 7 | 32 | 9 | 24 | 27 |
| | KLpl-15 | 56 | 9 | 45 | 29 | 36 | 29 |
| | KLpl-16 | 67 | 6 | 47 | 6 | 37 | 16 |
| | KLpl-17 | 60 | 11 | 16 | 1 | 210 | 13 |
| | KLpl-18 | 43 | 9 | 44 | 19 | 47 | 10 |
| | KLpl-19 | 52 | 4 | 42 | 14 | 62 | 23 |
| | KLpl-20 | 62 | 11 | 52 | 13 | 66 | 17 |
| | KLpl-21 | 67 | 11 | 57 | 12 | 66 | 13 |
| | KLpl-22 | 192 | 9 | 252 | 12 | 232 | 14 |
| | KLpl-23 | 157 | 14 | 214 | 15 | 248 | 31 |
| | KLpl-24 | 35 | 21 | 25 | 31 | 22 | 24 |
| | KLpl-25 | 7 | 19 | 17 | 15 | 23 | 14 |
| | KLpl-26 | 32 | 31 | 33 | 31 | 35 | 15 |
| | KLpl-27 | 24 | 27 | 26 | 29 | 21 | 17 |
| Lactobacillus | KLR-1 | 503 | 29 | 481 | 11 | 507 | 24 |
| reuteri | KLR-2 | 203 | 25 | 418 | 92 | 245 | 23 |
| | KLR-3 | 35 | 543 | 22 | 596 | 34 | 567 |
| | KLR-4 | 399 | 310 | 434 | 34 | 527 | 421 |
| | KLR-5 | 382 | 291 | 338 | 224 | 235 | 142 |
| | KLR-6 | 144 | 24 | 153 | 83 | 181 | 92 |
| | KLR-7 | 283 | 22 | 219 | 42 | 172 | 20 |
| | KLR-8 | 261 | 306 | 316 | 317 | 249 | 265 |
| | KLR-9 | 167 | 38 | 109 | 4 | 188 | 14 |
| | KLR-10 | 49 | 283 | 59 | 371 | 79 | 266 |
| | KLR-11 | 166 | 36 | 261 | 73 | 319 | 23 |
| | KLR-12 | 146 | 15 | 512 | 52 | 322 | 82 |
| | KLR-13 | 453 | 12 | 336 | 37 | 394 | 14 |
| | KLR-14 | 181 | 15 | 148 | 118 | 121 | 143 |
| | KLR-15 | 281 | 8 | 253 | 5 | 234 | 14 |
| | ZM-122 | 73 | 69 | 86 | 93 | 82 | 68 |
| Lactobacillus | KLrh-1 | 33 | 38 | 17 | 28 | 25 | 24 |
| rhamnosus | KLrh-2 | 14 | 11 | 6 | 9 | 6 | 7 |
| | KLrh-3 | 13 | 20 | 7 | 16 | 13 | 7 |
| | KLrh-4 | 14 | 2 | 7 | 10 | 7 | 2 |
| | KLrh-5 | 14 | 13 | 7 | 23 | 1 | 4 |
| | KLrh-6 | 10 | 7 | 7 | 10 | 6 | 8 |
| | KLrh-7 | 11 | 8 | 6 | 9 | 6 | 11 |
| | KLrh-8 | 41 | 8 | 37 | 10 | 26 | 9 |
| | KLrh-9 | 49 | 21 | 25 | 20 | 46 | 47 |
| | KLrh-10 | 58 | 20 | 6 | 7 | 53 | 49 |
| | KLrh-11 | 7 | 1 | 13 | 5 | 1 | 1 |
| Lactobacillus | KLsa-1 | 11 | 8 | 7 | 9 | 5 | 9 |
| salivarius | KLsa-2 | 9 | 6 | 6 | 8 | 4 | 9 |
| | KLsa-3 | 260 | 7 | 308 | 10 | 327 | 9 |
| | KLsa-4 | 10 | 7 | 7 | 10 | 7 | 9 |
| | KLsa-5 | 10 | 7 | 4 | 11 | 7 | 22 |
| | KLsa-6 | 12 | 8 | 7 | 11 | 6 | 9 |
| | KLsa-7 | 10 | 7 | 8 | 10 | 5 | 11 |
| | KLsa-8 | 17 | 14 | 8 | 11 | 6 | 11 |
| | KLsa-9 | 16 | 13 | 8 | 12 | 7 | 10 |
| | KLsa-10 | 15 | 12 | 6 | 12 | 7 | 10 |
| Bifidobacterium | KBla-11 | 17 | 0 | 12 | 13 | 1 | 14 |
| animalis | KBla-12 | 65 | 9 | 7 | 12 | 16 | 9 |
| | KBla-13 | 15 | 20 | 26 | 19 | 12 | 17 |
| | KBla-14 | 62 | 11 | 31 | 18 | 16 | 19 |
| | KBla-15 | 31 | 9 | 25 | 21 | 26 | 41 |
| | KBla-16 | 175 | 1 | 207 | 92 | 231 | 6 |
| | KBla-17 | 20 | 9 | 18 | 5 | 15 | 2 |
| | KBla-18 | 130 | 12 | 152 | 14 | 173 | 17 |
| | KBla-19 | 5 | 3 | 41 | 19 | 27 | 10 |
| | KBla-20 | 19 | 8 | 42 | 24 | 62 | 23 |
| | KBla-21 | 23 | 12 | 52 | 23 | 66 | 17 |
| | KBla-22 | 97 | 9 | 57 | 12 | 46 | 13 |
| | KBla-23 | 36 | 6 | 52 | 16 | 32 | 14 |
| | KBla-24 | 33 | 5 | 14 | 15 | 48 | 31 |
| | KBla-25 | 123 | 10 | 204 | 7 | 152 | 21 |
| | KBlo-1 | 5 | 8 | 6 | 12 | 3 | 5 |
| Bifidobacterium | KBlo-2 | 311 | 17 | 271 | 21 | 332 | 2 |
| longum | KBlo-3 | 9 | 39 | 12 | 21 | 14 | 20 |
| | KBlo-4 | 19 | 8 | 13 | 16 | 21 | 2 |
| | KBlo-5 | 37 | 14 | 25 | 5 | 21 | 11 |
| | KBlo-6 | 9 | 2 | 13 | 7 | 16 | 2 |
| Bifidobacterium | KBin-1 | 252 | 18 | 201 | 7 | 229 | 14 |
| infantis | | | | | | | |
| Pediococcus | KPac-1 | 270 | 4 | 311 | 11 | 287 | 21 |
| acidilactici | KPac-2 | 279 | 3 | 319 | 12 | 253 | 6 |
| | KPac-3 | 243 | 9 | 275 | 3 | 261 | 7 |

Through the screening by the purine precursor-degrading effect test, the following probiotic strains were obtained: probiotic strains with the advantage of degrading nucleosides (>100 mg/OD·h·L): Lactobacillus fermentum (KLfe-1, KLfe-5, KLfe-7, and KLfe-8), Lactobacillus reuteri (KLR-1, KLR-2, KLR-6, KLR-7, KLR-9, KLR-11, KLR-12, KLR-13, KLR-14, and KLR-15), Lactobacillus helveticus (KLhe-9), Lactobacillus plantarum (KLp1-22 and KLp1-23), Lactobacillus casei (KLca-9), Pediococcus acidilactici (KPac-1, KPac-2, and KPac-3), Lactobacillus bulgaricus (KLdB-1, KLdB-2, and KLdB-3), Bifidobacterium animalis (KB1a-16, KB1a-18, and KB1a-25), Bifidobacterium longum (KBlo-2), Bifidobacterium infantis (KBin-1), Lactobacillus acidophilus (KLac-2), Lactobacillus salivarius (KLsa-3); and probiotic strains with the advantage of degrading nucleotides (>100 mg/OD·h·L): Lactobacillus reuteri (KLR-3 and KLR-10).

Meanwhile, through the screening by the purine precursor-degrading effect test, the probiotic strains with the advantage of degrading both purine nucleosides and purine nucleotides (>100 mg/OD·h·L): Lactobacillus reuteri (KLR-4, KLR-5, and KLR-8).

The inosine-degrading capability of Lactobacillus gasseri (named PA-3) isolated and picked out from uric acid-reducing probiotic products of Meiji Co., Ltd., Japan as determined using the test method in this example, was relatively close to the results (about 600 pmol/min/L×10⁹ CFU) reported in the published literature (Naruomi Yamada, et. al., Lactobacillus gasseri PA-3 Uses the Purines IMP, Inosine and Hypoxanthine and Reduces Their Absorption in Rats, Microorganisms (2017), 5, 10). However, its inosine-degrading capability was much weaker than that of the Lactobacillus reuteri and Lactobacillus fermentum picked out by the present patent.

Dietary DNA will be digested and degraded into deoxynucleotides. Based on this, the dominant probiotic strains picked out were further screened by the degradation test using deoxyribonucleosides (deoxyguanosine and deoxyadenosine) and deoxyribonucleotides (deoxyadenosine monophosphate and deoxyguanosine monophosphate) as substrates. The test results are shown in Table 4. The results showed that the dominant probiotics for degrading the nucleosides/nucleotides also had a relatively strong capability of degrading deoxynucleotides/deoxynucleosides. Therefore, they can degrade dietary DNA and RNA after intestinal digestion, and thus reduce the absorption of food-derived purine precursors.

TABLE 4
Degradation test of deoxynucleosides and deoxynucleotides by probiotics

| | | Rate of degradation of purine precursors (mg/OD · h · L) | | | |
|---|---|---|---|---|---|
| | Strain | | Deoxyguanosine | | Deoxyadenosine |
| Strain name | code | Deoxyguanosine | monophosphate | Deoxyadenosine | monophosphate |
| Lactobacillus | KLac-2 | 312 | 25 | 284 | 17 |
| acidophilus | | | | | |
| Lactobacillus | KLca-2 | 151 | 18 | 248 | 39 |
| casei | | | | | |
| Lactobacillus | KLdB-1 | 168 | 12 | 196 | 15 |
| bulgaricus | KLdB-2 | 215 | 21 | 273 | 14 |
| | KLdB-3 | 114 | 25 | 163 | 17 |
| Lactobacillus | KLfe-1 | 283 | 29 | 332 | 25 |
| fermentum | KLfe-5 | 471 | 15 | 421 | 27 |
| | KLfe-7 | 413 | 35 | 467 | 66 |
| | KLfe-8 | 233 | 27 | 329 | 36 |
| | KLpl-22 | 151 | 29 | 182 | 11 |
| | KLpl-23 | 177 | 24 | 253 | 28 |
| Lactobacillus | KLhe-9 | 341 | 35 | 319 | 23 |
| helveticus | | | | | |
| Pediococcus | KPac-1 | 170 | 14 | 187 | 24 |
| acidilactici | KPac-2 | 229 | 13 | 216 | 8 |
| | KPac-3 | 263 | 11 | 221 | 13 |
| Lactobacillus | KLR-1 | 443 | 39 | 357 | 25 |
| reuteri | KLR-2 | 233 | 18 | 244 | 21 |
| | KLR-3 | 38 | 513 | 14 | 540 |
| | KLR-4 | 429 | 342 | 511 | 413 |
| | KLR-5 | 341 | 277 | 335 | 182 |
| | KLR-6 | 155 | 63 | 172 | 105 |
| | KLR-7 | 233 | 12 | 162 | 21 |
| | KLR-8 | 291 | 246 | 255 | 205 |
| | KLR-9 | 148 | 28 | 198 | 24 |
| | KLR-10 | 44 | 251 | 72 | 222 |
| | KLR-11 | 187 | 16 | 213 | 28 |
| | KLR-12 | 166 | 15 | 232 | 52 |
| | KLR-13 | 481 | 12 | 434 | 18 |
| | KLR-14 | 211 | 10 | 171 | 43 |
| | KLR-15 | 251 | 18 | 241 | 17 |
| Bifidobacterium | KBla-16 | 155 | 8 | 213 | 9 |
| animalis | KBla-18 | 135 | 22 | 163 | 47 |
| | KBla-25 | 163 | 19 | 150 | 31 |
| Bifidobacterium | KBlo-2 | 271 | 13 | 281 | 12 |
| longum | | | | | |
| Bifidobacterium | KBin-1 | 242 | 14 | 259 | 16 |
| infantis | | | | | |

### EXAMPLE 2. RESCREENING OF PROBIOTICS WITH CAPABILITY OF DEGRADING PURINE PRECURSORS UNDER A NUTRIENT-CONTAINING CONDITION

The human intestinal tract is a nutrient-rich environment, and the case where probiotics degrade purine nucleosides and nucleotides under a condition of a nutrient-free system containing only nucleoside and nucleotide substrates does not demonstrate that the probiotics still have degradation capability in a nutrient-rich intestinal tract. Therefore, it is very important to select for a screening condition that contains nutrients and does not affect the test. After screening and optimization, the inventors selected a nutrient-containing test system that can ensure the growth of bacteria and does not affect the test. The test system comprises the following components: 20 mM phosphate; 0.2% glucose, 0.25% yeast extract powder and 0.2% ammonium sulfate, pH 6.86. The dominant probiotic strains picked out in Example 1 were rescreened in the above nutrient-containing test buffer. The strains to be screened were adjusted to a concentration of OD₆₀₀=2.7, added to a reaction system (adjusted to a bacteria concentration of OD₆₀₀=0.3), and reacted for 3 h. At the end of the reaction, the OD₆₀₀ value was determined again to evaluate the proliferation of the probiotics during the reaction. After the reaction was completed, the reaction solution was centrifuged to collect the supernatant. 900 μL of the supernatant was taken and added with 100 μL of perchloric acid terminating buffer, and the mixture was filtered through a 0.22 μm filter and then detected for the degradation efficiency of the purine precursors by HPLC. Meanwhile, the rate of degradation was compared with that in Example 1 to evaluate the difference between the probiotic strains in a nutrient-free reaction system and those in a nutrient-containing reaction system. The results are shown in Tables 5-7.

TABLE 5
Growth status of probiotic strains in a nutrient-containing test system

| | OD value of | OD value | | OD value | OD value |
|---|---|---|---|---|---|
| | test zero | of test | | of test | of test |
| Strain | point | endpoint | Strain | zero point | endpoint |
| KLac-2 | 0.3 | 0.382 | KLR-1 | 0.3 | 0.673 |
| KLca-2 | 0.3 | 0.395 | KLR-2 | 0.3 | 0.461 |
| KLdB-1 | 0.3 | 0.421 | KLR-3 | 0.3 | 0.338 |
| KLdB-2 | 0.3 | 0.382 | KLR-4 | 0.3 | 1.339 |
| KLdB-3 | 0.3 | 0.361 | KLR-5 | 0.3 | 0.632 |
| KLfe-1 | 0.3 | 0.694 | KLR-6 | 0.3 | 0.538 |
| KLfe-5 | 0.3 | 0.397 | KLR-7 | 0.3 | 0.441 |
| KLfe-7 | 0.3 | 0.442 | KLR-8 | 0.3 | 0.517 |
| KLfe-8 | 0.3 | 0.512 | KLR-9 | 0.3 | 0.482 |
| KLpl-22 | 0.3 | 0.771 | KLR-11 | 0.3 | 0.301 |
| KLpl-23 | 0.3 | 0.742 | KLR-12 | 0.3 | 0.318 |
| KLhe-9 | 0.3 | 0.637 | KLR-13 | 0.3 | 0.611 |
| KPac-1 | 0.3 | 0.421 | KLR-14 | 0.3 | 0.324 |
| KPac-2 | 0.3 | 0.452 | KLR-15 | 0.3 | 0.752 |
| KPac-3 | 0.3 | 0.368 | KBla-16 | 0.3 | 0.356 |
| KBlo-2 | 0.3 | 0.337 | KBla-18 | 0.3 | 0.389 |
| KBin-1 | 0.3 | 0.401 | KBla-25 | 0.3 | 0.413 |

TABLE 6
Rate of degradation of purine precursors by probiotic strains in a nutrient-containing test system

| | | Rate of degradation of purine precursors (mg/OD · h · L) | | | | | |
|---|---|---|---|---|---|---|---|
| | | | Guanosine | | Adenosine | | Deoxyguanosine |
| Strain name | Strain code | Guanosine | monophosphate | Adenosine | monophosphate | Deoxyguanosine | monophosphate |
| Lactobacillus | KLac-2 | 224 | 15 | 234 | 15 | 201 | 23 |
| acidophilus | | | | | | | |
| Lactobacillus | KLca-2 | 116 | 14 | 243 | 36 | 132 | 16 |
| casei | | | | | | | |
| Lactobacillus | KLdB-1 | 246 | 12 | 244 | 15 | 189 | 20 |
| bulgaricus | KLdB-2 | 229 | 21 | 287 | 26 | 212 | 18 |
| | KLdB-3 | 122 | 25 | 142 | 36 | 103 | 17 |
| Lactobacillus | KLfe-1 | 265 | 8 | 365 | 15 | 234 | 21 |
| fermentum | KLfe-5 | 451 | 16 | 529 | 19 | 444 | 18 |
| | KLfe-7 | 173 | 1 | 283 | 56 | 213 | 13 |
| | KLfe-8 | 183 | 10 | 376 | 16 | 167 | 12 |
| Lactobacillus | KLpl-22 | 203 | 1 | 282 | 13 | 175 | 20 |
| plantarum | KLpl-23 | 211 | 7 | 262 | 27 | 191 | 14 |
| Lactobacillus | KLhe-9 | 281 | 3 | 353 | 17 | 243 | 5 |
| helveticus | | | | | | | |
| Pediococcus | KPac-1 | 277 | 4 | 307 | 23 | 205 | 18 |
| acidilactici | KPac-2 | 285 | 5 | 239 | 16 | 233 | 10 |
| | KPac-3 | 273 | 19 | 291 | 27 | 243 | 10 |
| Lactobacillus | KLR-1 | 519 | 1 | 571 | 14 | 461 | 9 |
| reuteri | KLR-2 | 417 | 13 | 238 | 27 | 303 | 15 |
| | KLR-3 | 41 | 524 | 33 | 572 | 29 | 463 |
| | KLR-4 | 468 | 359 | 543 | 448 | 431 | 360 |
| | KLR-5 | 251 | 182 | 153 | 126 | 281 | 202 |
| | KLR-6 | 47 | 27 | 87 | 12 | 55 | 41 |
| | KLR-7 | 177 | 18 | 126 | 26 | 197 | 11 |
| | KLR-8 | 241 | 236 | 194 | 159 | 262 | 182 |
| | KLR-9 | 218 | 26 | 175 | 4 | 155 | 24 |
| | KLR-10 | 45 | 343 | 49 | 346 | 41 | 277 |
| | KLR-11 | 164 | 43 | 392 | 12 | 153 | 26 |
| | KLR-12 | 179 | 19 | 352 | 37 | 157 | 16 |
| | KLR-13 | 581 | 13 | 449 | 32 | 455 | 14 |
| | KLR-14 | 202 | 23 | 118 | 134 | 213 | 10 |
| | KLR-15 | 378 | 11 | 243 | 12 | 276 | 8 |
| Bifidobacterium | KBla-16 | 141 | 3 | 264 | 26 | 125 | 12 |
| animalis | KBla-18 | 164 | 15 | 197 | 23 | 151 | 24 |
| | KBla-25 | 212 | 15 | 159 | 27 | 166 | 17 |
| Bifidobacterium | KBlo-2 | 255 | 28 | 332 | 2 | 224 | 19 |
| longum | | | | | | | |
| Bifidobacterium | KBin-1 | 453 | 12 | 359 | 21 | 299 | 26 |
| infantis | | | | | | | |

TABLE 7
Ratio of rate of degradation of purine precursors by probiotic strains in a nutrient-containing
medium to that in a nutrient-free medium

| | | Ratio of rate of degradation under a nutrient-containing condition to that | | | | | |
|---|---|---|---|---|---|---|---|
| | | under a nutrient-free condition | | | | | |
| | Strain | | Guanosine | | Adenosine | | Deoxyguanosine |
| Strain name | code | Guanosine | monophosphate | Adenosine | monophosphate | Deoxyguanosine | monophosphate |
| Lactobacillus | KLac-2 | 0.86 | 0.75 | 1.09 | 1.36 | 0.64 | 0.92 |
| acidophilus | | | | | | | |
| Lactobacillus | KLca-2 | 0.95 | 0.36 | 1.14 | 1.24 | 0.87 | 0.89 |
| casei | | | | | | | |
| Lactobacillus | KLdB-1 | 1.03 | 6.00 | 1.13 | 1.15 | 1.13 | 1.67 |
| bulgaricus | KLdB-2 | 1.05 | 0.91 | 1.13 | 1.44 | 0.99 | 0.86 |
| | KLdB-3 | 0.79 | 1.25 | 0.65 | 2.25 | 0.90 | 0.68 |
| Lactobacillus | KLfe-1 | 1.05 | 0.21 | 1.21 | 3.00 | 0.83 | 0.72 |
| fermentum | KLfe-5 | 0.99 | 0.80 | 1.29 | 2.71 | 0.94 | 1.20 |
| | KLfe-7 | 0.37 | 0.03 | 0.57 | 0.65 | 0.52 | 0.37 |
| | KLfe-8 | 0.86 | 0.38 | 1.22 | 2.67 | 0.72 | 0.44 |
| Lactobacillus | KLpl- | 1.06 | 0.11 | 1.22 | 0.93 | 1.16 | 0.69 |
| | 22 | | | | | | |
| plantarum | KLpl- | 1.34 | 0.50 | 1.06 | 0.87 | 1.08 | 0.58 |
| | 23 | | | | | | |
| Lactobacillus | KLhe-9 | 0.84 | 0.10 | 1.02 | 0.77 | 0.71 | 0.14 |
| helveticus | | | | | | | |
| Pediococcus | KPac-1 | 1.03 | 1.00 | 1.07 | 1.10 | 1.21 | 1.29 |
| acidilactici | KPac-2 | 1.02 | 1.67 | 0.94 | 2.67 | 1.02 | 0.77 |
| | KPac-3 | 1.12 | 2.11 | 1.11 | 3.86 | 0.92 | 0.91 |
| Lactobacillus | KLR-1 | 1.03 | 0.03 | 1.13 | 0.58 | 1.04 | 0.23 |
| reuteri | KLR-2 | 2.05 | 0.52 | 0.97 | 1.17 | 1.30 | 0.83 |
| | KLR-3 | 1.17 | 0.97 | 0.97 | 1.01 | 0.76 | 0.90 |
| | KLR-4 | 1.17 | 1.16 | 1.03 | 1.06 | 1.00 | 1.05 |
| | KLR-5 | 0.66 | 0.63 | 0.65 | 0.89 | 0.82 | 0.73 |
| | KLR-6 | 0.33 | 1.13 | 0.48 | 0.13 | 0.35 | 0.65 |
| | KLR-7 | 0.63 | 0.82 | 0.73 | 1.30 | 0.85 | 0.92 |
| | KLR-8 | 0.92 | 0.77 | 0.78 | 0.60 | 0.90 | 0.74 |
| | KLR-9 | 1.31 | 0.68 | 0.93 | 0.29 | 1.05 | 0.86 |
| | KLR-10 | 0.92 | 1.21 | 0.62 | 1.30 | 0.93 | 1.10 |
| | KLR-11 | 0.99 | 1.19 | 1.23 | 0.52 | 0.82 | 1.63 |
| | KLR-12 | 1.23 | 1.27 | 1.09 | 0.45 | 0.95 | 1.07 |
| | KLR-13 | 1.28 | 1.08 | 1.14 | 2.29 | 0.95 | 1.17 |
| | KLR-14 | 1.12 | 1.53 | 0.98 | 0.94 | 1.01 | 1.00 |
| | KLR-15 | 1.35 | 1.38 | 1.04 | 0.86 | 1.10 | 0.44 |
| Bifidobacterium | KBla- | 0.81 | 3.00 | 1.14 | 4.33 | 0.81 | 1.50 |
| animalis | 16 | | | | | | |
| | KBla- | 1.26 | 1.25 | 1.14 | 1.35 | 1.12 | 1.09 |
| | 18 | | | | | | |
| | KBla- | 1.72 | 1.50 | 1.05 | 1.29 | 1.02 | 0.89 |
| | 25 | | | | | | |
| Bifidobacterium | KBlo-2 | 0.82 | 1.65 | 1.00 | 1.00 | 0.83 | 1.46 |
| longum | | | | | | | |
| Bifidobacterium | KBin-1 | 1.80 | 0.67 | 1.57 | 1.50 | 1.24 | 1.86 |
| infantis | | | | | | | |

The results in Table 5 showed that the OD values of most of the strains in the nutrient-containing test buffer were increased, indicating that the nutrients in the culture medium can maintain the survival of the strains and enable the growth and proliferation of the probiotics. The results in Tables 6 and 7 showed that some of the probiotic strains (e.g., Lactobacillus bulgaricus strain KLdB-3; Lactobacillus fermentum strain KLfe-7; and Lactobacillus reuteri strain KLR-6) could degrade purines under a nutrient-free condition containing only nucleotides and nucleosides, but had a significantly weakened purine-degrading capability under a nutrient-containing condition (the rate of degradation of at least one substrate was decreased by ≥30%). The target strains preferred in the present application must have significant purine precursor-degrading capability under both test conditions, e.g., Lactobacillus reuteri strains KLR-1, KLR-3 and KLR-13.

Meanwhile, Lactobacillus reuteri strains KLR-5 and KLR-8 could degrade purines under a nutrient-free condition containing only nucleotides and nucleosides, but had a significantly weakened purine-degrading capability under a nutrient-containing condition (the rate of degradation of at least one substrate was decreased by >30%), while Lactobacillus reuteri strain KLR-4 had a significant purine precursor-degrading capability under both test conditions, and had even enhanced degradation capability in a nutrient-containing medium.

### EXAMPLE 3. SCREENING OF PURINE-DEGRADING CANDIDATE Lactobacillus STRAINS UNDER DIFFERENT pH CONDITIONS

The pH value of the human intestinal tract ranges from 5.5 (duodenum) to 7.5 (large intestine). In order to screen for probiotics capable of degrading purine precursors throughout the intestinal tract, the Lactobacillus reuteri strains picked out in Example 2 were used as candidates and tested for the ability for degrading purines under conditions simulating the pH (5.0-7.5) of the human intestinal tract. The test was performed using guanosine and adenosine as substrates. Since KLR-3 and KLR-mainly had activity for degrading nucleotides, the test substrates were guanosine monophosphate and adenosine monophosphate. The test results (Table 8-1) showed that the ability of Lactobacillus reuteri strains KLR-1 and KLR-13 for degrading guanosine and adenosine was relatively stable at different pH values, and the ability of Lactobacillus reuteri strain KLR-3 for degrading guanosine monophosphate and adenosine monophosphate was relatively stable at different pH values. In contrast, the ability of KLR-10 for degrading guanosine monophosphate and adenosine monophosphate fluctuated greatly at different pH values.

TABLE 8-1
Rate of degradation of purine precursors by Lactobacillus reuteri
strains at different pH values

| | Rate of degradation of guanosine at different pH values (mg/(OD · h · L)) | | | | | |
|---|---|---|---|---|---|---|
| Strain name | pH 5.0 | pH 5.5 | pH 6.0 | pH 6.5 | pH 7.0 | pH 7.5 |
| Lactobacillus reuteri | 541 | 533 | 547 | 529 | 540 | 559 |
| KLR-1 | | | | | | |
| Lactobacillus reuteri | 553 | 535 | 534 | 581 | 601 | 578 |
| KLR-3 (substrate is | | | | | | |
| guanosine | | | | | | |
| monophosphate) | | | | | | |
| Lactobacillus reuteri | 133 | 191 | 188 | 242 | 283 | 277 |
| KLR-10 (substrate is | | | | | | |
| guanosine | | | | | | |
| monophosphate) | | | | | | |
| Lactobacillus reuteri | 523 | 517 | 534 | 551 | 537 | 528 |
| KLR-13 | | | | | | |
| | Rate of degradation of adenosine at different pH values (mg/(OD · h · L)) | | | | | |
| Strain name | pH 5.0 | pH 5.5 | pH 6.0 | pH 6.5 | pH 7.0 | pH 7.5 |
| Lactobacillus reuteri | 545 | 538 | 537 | 556 | 548 | 550 |
| KLR-1 | | | | | | |
| Lactobacillus reuteri | 523 | 527 | 534 | 531 | 535 | 528 |
| KLR-3 (substrate is | | | | | | |
| adenosine | | | | | | |
| monophosphate) | | | | | | |
| Lactobacillus reuteri | 161 | 183 | 191 | 264 | 293 | 267 |
| KLR-10 (substrate is | | | | | | |
| adenosine | | | | | | |
| monophosphate) | | | | | | |
| Lactobacillus reuteri | 443 | 431 | 436 | 453 | 432 | 421 |
| KLR-13 | | | | | | |

Meanwhile, the Lactobacillus reuteri strain KLR-4 picked out in the Example 2 was used as a candidate and tested for the ability for degrading purines under conditions simulating the pH (5.0-7.5) of the human intestinal tract. The test was performed using guanosine and guanosine monophosphate as well as adenosine and adenosine monophosphate as substrates. The test results (Table 8-2) showed that the ability of Lactobacillus reuteri strain KLR-4 for degrading guanosine and guanosine monophosphate as well as adenosine and adenosine monophosphate was relatively stable at different pH values.

TABLE 8-2
Rate of degradation of purine precursors by Lactobacillus reuteri
strain KLR-4 at different pH values

| | Rate of degradation of nucleosides at different | | | | | |
|---|---|---|---|---|---|---|
| | pH values (mg/(OD · h · L)) | | | | | |
| Substrate | pH5.0 | pH5.5 | pH6.0 | pH6.5 | pH7.0 | pH7.5 |
| guanosine | 364 | 371 | 398 | 369 | 365 | 359 |
| adenosine | 459 | 494 | 488 | 522 | 483 | 472 |
| guanosine | 345 | 348 | 357 | 366 | 340 | 359 |
| monophosphate | | | | | | |
| adenosine | 423 | 422 | 434 | 431 | 445 | 434 |
| monophosphate | | | | | | |

### EXAMPLE 4. IDENTIFICATION OF PRODUCTS OBTAINED AFTER THE DEGRADATION OF PURINE PRECURSORS BY PROBIOTICS

The identification and analysis of the products were performed by liquid chromatography-mass spectrometry (Thermo Scientific Q Exactive) according to the peaks of the products obtained after the degradation of various purine precursor substrates in Examples 1-3. The HPLC conditions were the same as those in Example 1, and the mass spectrometry conditions were as follows: Spray Voltage: 3200V; Capillary Temperature: 300.00° C.; Sheath Gas: 40.00 L/min; Aux Gas: 15.00 L/min; Max Spray Current: 100.00 mA; Probe Heater Temp: 350.00° C.; S-Lens RF Level: 50.00° C. Ion Source: ESI-ms.

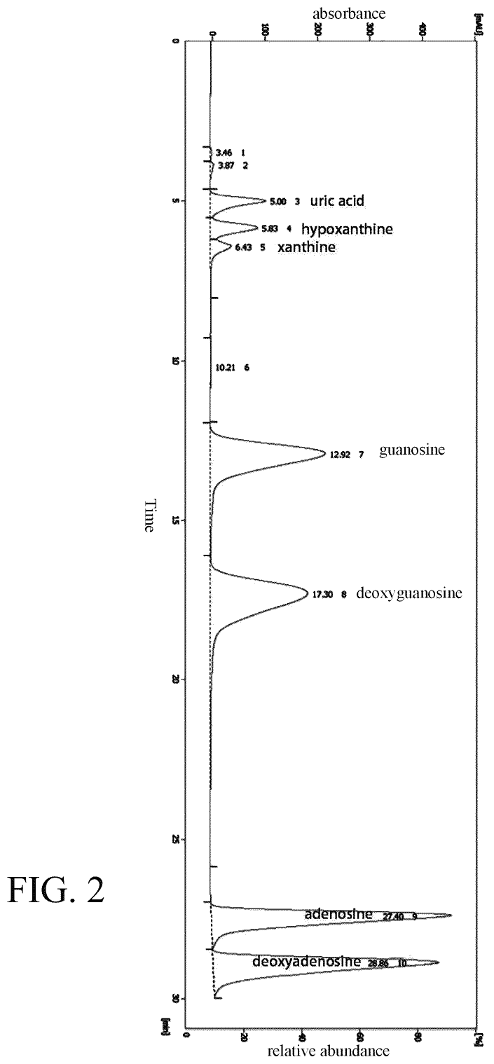

The standard substance was a mixture consisting of uric acid, hypoxanthine, xanthine, deoxyguanosine, deoxyadenosine, guanosine and adenosine (FIG. 2). The mass spectrometry results showed that the molecular weight was well consistent with the theoretical molecular weight of each standard substance. Test samples: samples obtained after guanosine, deoxyguanosine, adenosine and deoxyadenosine substrates were subjected to a reaction with the KLR-1 strain for 2 h; samples obtained after guanosine monophosphate, deoxyguanosine monophosphate, adenosine monophosphate and deoxyadenosine monophosphate substrates were subjected to a reaction with the KLR-3 strain for 2 h and centrifuged to remove the bacteria; and samples obtained after guanosine, deoxyguanosine, guanosine monophosphate, deoxyguanosine monophosphate, adenosine, deoxyadenosine, adenosine monophosphate and deoxyadenosine monophosphate substrates were subjected to a reaction with the KLR-4 strain for 2 h and centrifuged to remove the bacteria. The results of identification of the degradation products by mass spectrometry are shown in Table 9. The results showed that after the purine precursor substrates were degraded by the probiotics, the main products were the corresponding purine bases, and a small amount of nucleosides/deoxynucleosides were also found in the reaction products of nucleotides/deoxynucleotides. It is speculated that the degradation process of purine precursors by the probiotics comprises firstly degrading the nucleotides or deoxynucleotides into nucleosides or deoxynucleosides, and then further degrading the nucleosides or deoxynucleosides into the corresponding purine bases. The solubility of the final products (purine bases) is greatly reduced, and the absorbable concentration of food-derived purine precursors in the gastrointestinal tract can be greatly reduced, so that the absorption of the purine precursors in food is reduced.

TABLE 9
Identification analysis of products obtained after the degradation
of purine precursors by probiotics

| Substrate | Final product |
|---|---|
| guanosine | guanine |
| deoxyguanosine | guanine |
| guanosine monophosphate | guanine |
| deoxyguanosine monophosphate | guanine |
| adenosine | adenine |
| deoxyadenosine monophosphate | adenine |
| adenosine monophosphate | adenine |
| deoxyadenosine monophosphate | adenine |

### EXAMPLE 5. TEST ON THE TOLERANCE OF CANDIDATE PROBIOTICS TO GASTROINTESTINAL TRACT

MRS liquid medium at pH 2.0, pH 3.0 and pH 4.0 were separately prepared for the test on the gastric acid tolerance of the candidate Lactobacillus reuteri strains, MRS liquid medium containing 0.1%, and 0.3% bile salts were separately prepared for the test on the bile salt tolerance of the candidate Lactobacillus reuteri strains, and the controls therefor were MRS liquid medium with pH not adjusted or MRS liquid medium with no bile salts added, respectively. The strains were inoculated into the test medium at an inoculum size of 1.0%, and cultured at 37° C. The culture medium was taken at time points 0 h, 2 h, 4 h and 6 h, and determined for viable count. The test was repeated twice. The test results are shown in Tables 10 and 11. The results showed that the candidate Lactobacillus reuteri strains KLR-1, KLR-3, KLR-4 and KLR-13 had good gastric acid tolerance and bile salt tolerance.

### (1) Gastric Acid Tolerance Test

TABLE 10
Results of gastric acid tolerance test of Lactobacillus reuteri
strains at 6 h

| | | | | Control (MRS |
|---|---|---|---|---|
| | | | | medium not |
| strain | pH2.0 | pH 3.0 | pH 4.0 | adjust pH) |
| KLR-1 | 2.8 * 10⁷ | 2.4 * 10⁸ | 7.4 * 10⁸ | 6.6 * 10⁸ |
| KLR-3 | 8.8 * 10⁶ | 1.1 * 10⁸ | 6.4 * 10⁸ | 7.4 * 10⁸ |
| KLR-4 | 3.5 * 10⁶ | 3.5 * 10⁸ | 6.4 * 10⁸ | 7.4 * 10⁸ |
| KLR-13 | 4.4 * 10⁷ | 2.3 * 10⁸ | 7.1 * 10⁸ | 8.6 * 10⁸ |

The results of 6 h of culture at pH 2.0 and pH 3.0 (as shown in Table 10) showed that the lower the pH was, the faster the viable count decreased; at pH 2.0, the viable count decreased significantly (by 95% or more); at pH 3.0, the viable count decreased but not changed over an order of magnitude; and in the medium at pH 4.0, the viable count did not significantly decrease along with the prolonging of time, indicating that the strains picked out in the present invention have better gastric acid tolerance.

### (2) Bile Salt Tolerance Test

TABLE 11
Results of bile salt tolerance test of Lactobacillus reuteri strains at 6 h

| | | | | Control (MRS |
|---|---|---|---|---|
| | | | | medium not |
| strain | 0.1% bile salt | 0.2% bile salt | 0.3% bile salt | add bile salt) |
| KLR-1 | 1.5 * 10⁸ | 4.6 * 10⁷ | 5.3 * 10⁵ | 6.9 * 10⁸ |
| KLR-3 | 2.7 * 10⁸ | 6.7 * 10⁷ | 5.9 * 10⁶ | 7.3 * 10⁸ |
| KLR-4 | 3.1 * 10⁸ | 4.3 * 10⁷ | 7.9 * 10⁵ | 7.3 * 10⁸ |
| KLR-13 | 1.6 * 10⁸ | 7.1 * 10⁷ | 8.3 * 10⁵ | 6.4 * 10⁸ |

The result of 6 h of culture containing 0.1%, 0.2% and 0.3% bile salts showed that the higher the concentration of the bile salt was, the faster the viable count decreased; the viable count was decreased by 3 orders of magnitude after 6 h of culture in a medium containing 0.3% bile salt, and there was basically no decrease in the number of colonies in a medium containing 0.1% bile salt, indicating that the strains picked out in the present invention have better tolerance to 0.1% bile salt. After the probiotic strains picked out in the present invention are orally administered and destroyed by gastric acid and bile salt, there was still relatively high number of viable bacteria to enter the intestinal tract to play a role.

### EXAMPLE 6. IDENTIFICATION OF GROWTH CHARACTERISTICS OF CANDIDATE Lactobacillus reuteri STRAINS

The candidate Lactobacillus reuteri strains picked out were biochemically identified using Lactobacillus biochemical identification strips (comprising aesculin, cellobiose, maltose, mannitol, salicin, sorbitol, sucrose, raffinose, inulin, lactose and hippuric acid, purchased from Qingdao Hope Bio-Technology Co., Ltd.) according to the method of GB4789.35. The specific procedures were as follows: single colonies were picked up from a purified plate using an inoculating needle and added to 2 mL of sterile normal saline, and pipetted and mixed well to obtain a bacterial suspension; the biochemical identification strip was taken out and added with the bacterial suspension at 100 μL/well after the covering film was tore off, and the mixture was mixed well, covered with a lid, put in a bottom bracket, cultured in an anaerobic incubator at 37° C. for 24-48 h, put on a recording card for observation after the culture was completed, and determined according to the description of the instructions. The identification results are shown in Table 12.

TABLE 12
Identification of growth characteristics of Lactobacillus reuteri strains

| | | Lactobacillus | Lactobacillus | Lactobacillus | Lactobacillus |
|---|---|---|---|---|---|
| | | reuteri KLR-1 | reuteri KLR-3 | reuteri KLR-4 | reuteri KLR-13 |
| Culture conditions | | 37° C., | 37° C., | 37° C., | 37° C., |
| | | facultative | facultative | facultative | facultative |
| | | anaerobic | anaerobic | anaerobic | anaerobic |
| Colony morphology | | Rod-shaped, | Rod-shaped, | Rod-shaped, | Rod-shaped, |
| | | single, paired | single, paired | single, paired | single, paired |
| | | or small | or small | or small | or small |
| | | cluster forms | cluster forms | cluster forms | cluster forms |
| Gram staining | | Gram positive | Gram positive | Gram positive | Gram positive |
| Lactic fermentation form | | Heterolactic | Heterolactic | Heterolactic | Heterolactic |
| | | fermentation | fermentation | fermentation | fermentation |
| Hydrogen peroxide test | | Negative | Negative | Negative | Negative |
| Aerobic growth | | + | + | + | + |
| Growth temperature | | 25-45° C. | 25-45° C. | 25-45° C. | 25-45° C. |
| Saccharide | aesculin | + | + | + | + |
| fermentability | cellobiose | + | − | − | + |
| | maltose | + | + | + | + |
| | mannitol | − | − | − | + |
| | salicin | + | − | − | + |
| | sorbitol | − | − | − | + |
| | sucrose | + | + | + | + |
| | raffinose | + | + | + | + |
| | inulin | + | − | − | + |
| | lactose | + | + | + | + |
| | hippuric | − | − | − | − |
| | acid | | | | |

| | | | | |
|---|---|---|---|---|
| Gas production | + | + | + | + |

### EXAMPLE 7. EFFECT OF ORAL ADMINISTRATION OF RECOMBINANT STRAINS ON SERUM URIC ACID OF RATS

### (1) Establishment of Hyperuricemia Animal Model

Eighty-four male SD rats weighing about 100 g were randomly divided into 14 groups with 6 rats in each group. After 3 days of acclimatized feeding, the modeling was started. Six rats in the blank group were fed with food normally and fed with water at 30 mL/24 h, and injected with normal saline intraperitoneally; the modeling control group and probiotic test groups were fed with food normally and fed with 20% yeast extract powder solution instead of water at 30 mL/24 h, and injected with potassium oxonate (250 mg/(kg/d)) intraperitoneally. Continuous feeding was performed for 5 days to construct a hyperuricemia model (modeling period), and blood was collected from the tail every 24 h for the last three days and detected for serum uric acid using a uric acid detection kit from Wuhan Life Origin Biotech Joint Stock Co., Ltd. The detection results are shown in Table 13. The results showed that a relatively stable hyperuricemia animal model was obtained.

### (2) Verification of Uric Acid-Reducing Effect of Probiotic Strains

The candidate Lactobacillus reuteri strains picked out in Example 5 (Lactobacillus reuteri KLR-1, Lactobacillus reuteri KLR-3, Lactobacillus reuteri KLR-4, and Lactobacillus reuteri KLR-13) were cultured in MRS medium at 37° C. for about 8-12 h (in late stage of logarithmic growth), and centrifuged at 12000 rpm to collect the bacteria. The bacteria were washed 3 times with sterile normal saline, weighed for the wet weight, adjusted to viable counts of about 5×10⁸ CFU/mL, 5×10⁹ CFU/mL, and 5×10¹⁰ CFU/mL with sterile normal saline. Lactobacillus reuteri strains KLR-1, KLR-3 and KLR-13 were mixed in a ratio and adjusted to total viable counts of about 5×10⁸ CFU/mL, 5×10⁹ CFU/mL, and 5×10¹⁰ CFU/mL. Meanwhile, Lactobacillus reuteri strains KLR-1 and KLR-3 were mixed in a ratio of 1:1 and adjusted to total viable counts of 5×10⁸ CFU/mL, 5×10⁹ CFU/mL, and 5×10¹⁰ CFU/mL. After being mixed well, the probiotic solutions were administered intragastrically to the established hyperuricemia rat model, at 1 mL/rat for the test group, twice every day. The rats were administered intragastrically for 7 consecutive days for the treatment (treatment period), and blood was collected from the tail every 24 h for the last 3 days and detected for serum uric acid. The results are shown in Table 13. The results showed that the serum uric acid level could be reduced by oral administration of Lactobacillus reuteri strains at different doses and of different strains and mixtures of Lactobacillus reuteri strains (KLR-1, KLR-3 and KLR-13 mixed in a ratio of 3:5:2), but there were differences. Among single probiotic strains, KLR-3 outperformed KLR-1 and KLR-13 at the same dose, indicating that degradation of nucleotides is more efficient than degradation of nucleosides, which may be consistent with the conclusion that the solubility of nucleotides is higher than that of nucleosides. Under the condition of the same variable count, the effect of KLR-4 was superior to that of KLR-1 or KLR-3, and was comparable to that of the mixture of the latter two, indicating that both degradations of nucleosides and nucleotides are very important in reducing the absorption of food-derived nucleic acids. The effect of the mixtures of strains was superior to that of single strains, indicating that both degradations of nucleosides and nucleotides are very important in reducing the absorption of food-derived nucleic acids.

TABLE 13
Change in concentration of serum uric acid (μmol/L)

| | Normal feeding period | Modeling period | Efficacy evaluation period |
|---|---|---|---|
| | (mean ± standard | (mean ± standard deviation | (mean ± standard deviation |
| Groups | deviation of 3 days) | of the last 3 days) | of the last 3 days) |
| Control | 114.10 ± 13.65 | 111.80 ± 8.86 | 113.87 ± 14.15 |
| Modeling control | 118.43 ± 21.34 | 190.80 ± 12.47 | 186.62 ± 28.55 |
| KLR-1(low-dose) | 119.90 ± 7.48 | 194.44 ± 29.46 | 171.99 ± 20.84* |
| KLR-1(mid-dose) | 121.67 ± 10.21 | 196.53 ± 15.65 | 164.21 ± 15.36* |
| KLR-1(high-dose) | 119.77 ± 13.68 | 197.41 ± 23.55 | 157.62 ± 13.65# |
| KLR-3(low-dose) | 120.75 ± 20.18 | 192.36 ± 3.42 | 161.73 ± 14.16* |
| KLR-3(mid-dose) | 121.57 ± 21.46 | 201.36 ± 8.78 | 155.12 ± 19.85# |
| KLR-3(high-dose) | 115.50 ± 19.25 | 198.79 ± 10.58 | 142.75 ± 13.06# |
| KLR-4(low-dose) | 122.4 ± 17.22 | 197.41 ± 23.66 | 152.15 ± 18.06# |
| KLR-4(mid-dose) | 120.81 ± 11.17 | 195.58 ± 18.63 | 145.43 ± 22.61# |
| KLR-4(high-dose) | 123.62 ± 18.71 | 199.64 ± 28.54 | 138.82 ± 27.55# |
| KLR-13(low-dose) | 124.45 ± 21.49 | 198.69 ± 10.56 | 176.18 ± 16.56* |
| KLR-13(mid-dose) | 120.31 ± 14.12 | 202.21 ± 15.24 | 169.64 ± 25.45* |
| KLR-13(high-dose) | 119.50 ± 2.70 | 207.66 ± 24.91 | 158.75 ± 32.69# |
| mixtures of | 121.77 ± 22.48 | 209.88 ± 24.41 | 147.53 ± 18.86# |
| Lactobacillus | | | |
| reuteri (low-dose) | | | |
| mixtures of | 120.31 ± 20.32 | 211.12 ± 18.21 | 138.16 ± 18.81# |
| Lactobacillus | | | |
| reuteri (mid-dose) | | | |
| mixtures of | 121.50 ± 18.22 | 214.79 ± 14.83 | 130.74 ± 16.64# |
| Lactobacillus | | | |
| reuteri (high-dose) | | | |

Note:
*p < 0.05 vs. the modeling control group; #p < 0.01 vs. the modeling control group

### EXAMPLE 8. EFFECT OF ORAL ADMINISTRATION OF lactobacillus reuteri STRAINS ON SERUM URIC ACID OF HYPERURICEMIA PATIENTS

The candidate Lactobacillus reuteri probiotic strains (KLR-1, KLR-3 and KLR-4) were produced as different kinds of probiotic solid beverages (KLR-1 and KLR-3 mixed in a ratio of 1:1 in the group of a solid beverage containing a mixture of Lactobacillus reuteri strains) in a factory meeting the probiotic production standard, wherein the viable count of the low-dose products was about 5×10⁹ CFU/bag, and the viable count of the high-dose products was about 5×10¹⁰ CFU/bag. After the production, the products were stored at −20° C. or 4° C. to ensure the viability of the probiotic powder during storage. Ninety hyperuricemia patients (serum uric acid >420 μmol/L) were recruited as volunteers, randomly divided into 9 groups with 10 patients in each group, and administered with the Lactobacillus reuteri strain KLR-1 (low-dose and high-dose), Lactobacillus reuteri strain KLR-3 (low-dose and high-dose), Lactobacillus reuteri strain KLR-4 (low-dose and high-dose), a mixture of Lactobacillus reuteri strains (low-dose and high-dose), respectively, once a bag and twice a day, and the intervention period was 30 days. The serum uric acid level was measured 3 consecutive days before the intervention and 28 ^{th}-30^{th} days of intervention period to evaluate the effect of the intervention.

TABLE 14
hange in concentration of serum uric acid

| | Before intervention | After intervention | Change values of serum |
|---|---|---|---|
| | (mean ± standard | (mean ± standard | uric acid (mean ± standard |
| Serum uric acid μmol/L | deviation) | deviation) | deviation) |
| Control | 583 ± 61 | 586 ± 79 | 3 ± 21 |
| KLR-1 (low-dose) | 573 ± 56 | 520 ± 58 | −54 ± 64* |
| KLR-1 (high-dose) | 612 ± 64 | 514 ± 54 | −98 ± 68# |
| KLR-3 (low-dose) | 596 ± 49 | 524 ± 50 | −71 ± 72* |
| KLR-3 (high-dose) | 623 ± 48 | 503 ± 53 | −120 ± 66# |
| KLR-4 (low-dose) | 614 ± 53 | 497 ± 44 | −117 ± 49▴ |
| KLR-4 (high-dose) | 620 ± 55 | 434 ± 47 | −186 ± 57#▪ [symbol] |
| mixture of Lactobacillus | 602 ± 69 | 481 ± 63 | −121 ± 77▴ |
| reuteri strains (low-dose) | | | |
| mixture of Lactobacillus | 616 ± 61 | 406 ± 51 | −209 ± 45#▪● |
| reuteri strains (high- | | | |
| dose) | | | |

Note:
*p < 0.05 vs. a blank control group; #p < 0.01 vs. a blank control group; ▴p < 0.05 vs. a KLR-1 group at an equal dose; ▪p < 0.01 vs. a KLR-1 group at an equal dose; [symbol] p < 0.05 vs. a KLR-3 group at an equal dose; ●p < 0.01 vs. a KLR-3 group at an equal dose; ⋄p < 0.05 vs. a group of a mixture of Lactobacillus reuteri strains at an equal dose; and ♦p < 0.01 vs. a group of a mixture of Lactobacillus reuteri strains at an equal dose.

The results of the human clinical trial showed that the serum uric acid-reducing effect of the Lactobacillus reuteri strain KLR-3 was superior to that of the Lactobacillus reuteri strain KLR-1 (low-dose: 71 vs. 54; high-dose: 120 vs. 98), indicating that the effect of degradation of purine nucleotides is better than that of degradation of purine nucleosides; under the condition of the equal viable count, the serum uric acid-reducing effect of the mixed powder of Lactobacillus reuteri strains was better than that of the single Lactobacillus reuteri strain KLR-1 or the single Lactobacillus reuteri strain KLR-3, and increased along with the increase of the viable count. The serum uric acid-reducing effect of the Lactobacillus reuteri strain KLR-4 was superior to that of the Lactobacillus reuteri strain KLR-1 (low-dose: 117 vs. 54; high-dose: 186 vs. 98) or that of the Lactobacillus reuteri strain KLR-3 (low-dose: 117 vs. 71; high-dose: 186 vs. 120), and increased along with the increase of the viable count; and the serum uric acid-reducing effect of the KLR-4 was close to that of the mixture of Lactobacillus reuteri strains (the mixture of KLR-1 and KLR-3) at an equal dose. The trial results indicate that the degradation of both of nucleosides and nucleotides are very important during in the reduction of the absorption of food-derived purine precursors, especially the degradation of nucleotides. Meanwhile, the effect of oral probiotics at different doses indicates that a large percentage of viable bacteria will be killed when the probiotics pass through the stomach, and probiotics at a high dose are helpful for increasing the number of the viable bacteria entering the intestinal tract, thereby achieving a better effect. Therefore, the viable count is an important factor for the efficacy of the oral probiotic products.

### EXAMPLE 9. PREPARATION OF PURINE-DEGRADING PROBIOTIC YOGURT POWDER PRODUCT

Yogurt is a healthy food containing probiotics that is widely favored by consumers. In this example, a simple preparation method for a yogurt powder product with the function of reducing serum uric acid and an operation process for fermenting yogurt thereof were proposed. The viable count of the yogurt powder product is ≥1×10⁵ CFU/g, and the formula per serving (about 250 g) is as follows: 180 g of full cream milk powder, 35 g of xylitol, 10 g of fructo-oligosaccharide, 10 g of resistant dextrin, 15 g of fruit powder, and 1 mg of lyophilized powder of Lactobacillus reuteri strain KLR-1 (viable count of 1×10¹¹ CFU/g), 1 mg of lyophilized powder of Lactobacillus reuteri strain KLR-3 (viable count of 1×10¹¹ CFU/g), or 2 mg of lyophilized powder of Lactobacillus reuteri strain KLR-4 (viable count of 1×10¹¹ CFU/g). One serving of the above yogurt powder product was poured into a yogurt jar, and about 800 mL of purified water or cooled boiled water was added. The mixture was stirred until it was completely dissolved, and added with water continuously to 1 L of scale mark. The jar was put into a yogurt machine and the yogurt was fermented at 38-40° C. for 8-12 h until it was solidified and ready to serve. It tastes better after being refrigerated at 4° C.

### EXAMPLE 10. PREPARATION OF A CHEWABLE TABLET CONTAINING PURINE-DEGRADING PROBIOTICS

In this example, a preparation method for a chewable tablet containing purine-degrading probiotics was provided. The formula of the chewable tablet is as follows: 40% isomaltulose, 23% citrus powder, 20% mixture of lyophilized powders of Lactobacillus reuteri strains KLR-1, KLR-3 and KLR-13 (1×10¹² CFU/g) or 20% lyophilized powder of Lactobacillus reuteri strain KLR-4 (1×10¹² CFU/g); 12% carboxymethylcellulose and 5% magnesium stearate. The materials were separately crushed and sieved through a 60-mesh sieve for later use. The corresponding materials were weighed according to the formula and mixed well, and the mixed material was poured into a feeding barrel of a tablet press for tableting, with the stamping pressure adjusted to ensure that the hardness of the probiotic chewable tablet was 10-15 kg and the weight of the tablet was about 2 g/tablet. The tablets were subpackaged into double blister packs or high-density polyethylene bottles (a desiccant bag needs to be added into the high-density polyethylene bottle) in a clean environment. The viable count of the chewable tablet product is ≥1×10⁸ CFU/g.

### EXAMPLE 11. PREPARATION OF AN ENTERIC-COATED PELLET CONTAINING PURINE-DEGRADING PROBIOTICS

In this example, a preparation method for an enteric-coated pellet containing purine-degrading probiotics was provided. The specific process is as follows: 30% lyophilized powder of Lactobacillus reuteri strain KLR-13 (1.5×10¹² CFU/g), 30% lyophilized powder of KLR-3 (1.5×10¹² CFU/g) or 60% lyophilized powder of Lactobacillus reuteri strain KLR-4 (1.5×10¹² CFU/g) was dissolved in sunflower seed oil to obtain a suspension with the probiotic powder content of 30%-40%. The probiotic-containing oil solution (core) and the enteric gum coating material (containing carrageenan, sodium alginate, gelatin, pullulan, calcium chloride and the like) were canned into 3 layers of enteric-coated pellets by a multilayer pellet machine. After air-cooling and drying at 25° C., the products were packaged into a waterproof polyethylene aluminum foil bag. The viable count of lactic acid bacteria of the pellet product is ≥1×10⁷ CFU/g.

### EXAMPLE 12. PREPARATION OF AN ENTERIC-COATED CAPSULE CONTAINING PURINE-DEGRADING PROBIOTICS

In this example, a preparation method for an enteric-coated capsule containing purine-degrading probiotics was provided. The specific formula is as follows: 35% fructo-oligosaccharide, 30% lyophilized powder of Lactobacillus reuteri strain KLR-1 (1.5×10¹² CFU/g), 30% lyophilized powder of KLR-3 (1.5×10¹² CFU/g), or 60% lyophilized powder of Lactobacillus reuteri strain KLR-4 (1.7×10¹² CFU/g) and 5% magnesium stearate. The above materials were separately sieved through a 60-mesh sieve for later use. The corresponding materials were weighed according to the formula, mixed well, filled into enteric-coated empty capsule shells, and encapsulated with a double-layer aluminum-plastic blister pack. The viable count of the capsule product is ≥5×10¹¹ CFU/g.

### EXAMPLE 13. PREPARATION OF A MILK BEVERAGE CONTAINING PURINE-DEGRADING PROBIOTICS

In this example, a preparation method for a milk beverage containing purine-degrading probiotics was provided. The specific process is as follows: activated Lactobacillus reuteri strains KLR-1, KLR-3, KLR-4 and KLR-13 were separately inoculated into a fermentation medium (4% glucose, 2% fructo-oligosaccharide, 3% whey protein, 2% yeast powder, 0.2% sodium citrate, 0.2% ammonium sulfate and 0.05% L-cysteine) that has been sterilized and cooled to 37° C., and fermented at 37° C. for 8 h to obtain Lactobacillus stock solutions. The three Lactobacillus stock solutions were mixed in a ratio of 3:5:2, and adjusted to a viable count of 4×10⁶−8×10⁸ CFU/g with sterile water, or adjusted to a viable count of 4×10⁶−8×10⁸ CFU/g for the Lactobacillus KLR-4 stock solution with sterile water. The solution was added with 6% skimmed milk powder, 7% edible glucose and 2% apple pectin, and adjusted to pH 3.5-3.8 and a viable count of ≥1×10⁶ CFU/g with citric acid and sodium citrate. The product was canned into sterile beverage bottles, subjected to thermoplastic sealing, and transported and stored at 4° C.

### EXAMPLE 14. PREPARATION OF ORAL COMPOSITION FOR DEGRADING URIC ACID AND DISSOLVING URATE CRYSTALS

Lyophilized probiotic powders containing Lactobacillus reuteri (viable count of 1.2×10⁹−2.1×10¹² CFU/g), Lactobacillus fermentum (viable count of 1.1×10⁹−2.0×10¹² CFU/g), Bacillus coagulans (viable count of 1.3×10⁸−1.5×10¹¹ CFU/g), Lactobacillus rhamnosus (viable count of 1.3×10⁹−2.1×10¹² CFU/g), Lactobacillus casei (viable count of 1.0×10⁹−1.4×10¹² CFU/g) and Lactobacillus plantarum (viable count of 5×10⁹−1.2×10¹² CFU/g), respectively, were combined with prebiotics (inulin, xylo-oligosaccharide, stachyose, etc.) and citrate. The formula of active ingredients thereof is shown in Table 15.

TABLE 15
Formula of oral compositions for dissolving urate crystals
Proportion of active ingredients

| | Proportion of active ingredients | | | | | | |
|---|---|---|---|---|---|---|---|
| | Composition | Composition | Composition | Composition | Composition | Composition | Composition |
| | 1 | 2 | 3 | 4 | 5 | 6 | 7 |
| Lactobacillus | 0.1 g | / | 0.1 g | / | / | 0.1 g | / |
| reuteri KLR-1 | | | | | | | |
| Lactobacillus | 0.1 g | / | 0.1 g | 0.4 g | / | 0.1 g | / |
| reuteri KLR-3 | | | | | | | |
| Lactobacillus | 0.1 g | / | 0.1 g | / | 0.9 g | 0.1 g | / |
| reuteri KLR-4 | | | | | | | |
| Lactobacillus | 0.1 g | / | 0.1 g | / | / | 0.1 g | / |
| reuteri KLR-13 | | | | | | | |
| Lactobacillus | 0.1 g | / | 0.1 g | / | / | 0.1 g | / |
| fermentum | | | | | | | |
| Bacillus | 0.1 g | / | 0.1 g | / | / | 0.1 g | / |
| coagulans | | | | | | | |
| Lactobacillus | 0.1 g | / | 0.1 g | 0.3 g | / | 0.1 g | / |
| rhamnosus | | | | | | | |
| Lactobacillus | 0.1 g | / | 0.1 g | / | / | 0.1 g | / |
| casei | | | | | | | |
| Lactobacillus | 0.1 g | / | 0.1 g | / | / | 0.1 g | / |
| plantarum | | | | | | | |
| inulin | / | 4.0 g | 4.0 g | / | 4.0 g | 4.0 g | / |
| xylo- | / | 2.0 g | 2.0 g | 5.8 g | / | 2.0 g | / |
| oligosaccharide | | | | | | | |
| stachyose | / | 1.2 g | 1.2 g | / | 3.0 g | 1.2 g | / |
| Citric acid | / | / | / | / | / | 0.3 g | 0.3 g |
| potassium citrate | / | / | / | 0.7 g | / | 0.3 g | 0.3 g |
| Calcium citrate | / | / | / | / | / | 0.3 g | 0.3 g |
| Magnesium | / | / | / | / | 1.2 g | 0.3 g | 0.3 g |
| citrate | | | | | | | |
| Potassium sodium | / | / | / | 0.8 g | / | 0.3 g | 0.3 g |

hydrogen citrate

### EFFECT VERIFICATION

### 1. Observation of Oral Compositions for Dissolving Urate Crystals and Tophi for Serum Uric Acid-Reducing Effect

120 patients with chronic gouty arthritis (gout history of 3-15 years, frequency of attacks ≤20 times/year) were recruited as volunteers and randomly divided into 8 groups with 15 patients in each group. The intervention for the groups were separately as follows: no intervention (control group), composition 1 intervention, composition 2 intervention, composition 3 intervention, composition 4 intervention, composition 5 intervention, composition 6 intervention, and composition 7 intervention, with an intervention period of 90 days, in the same manner: supplementing with excipients (e.g. maltodextrin, corn starch and microcrystalline cellulose) to enable each composition to reach the weight of 10 g, taken with warm water below 42° C. 1-2 h after breakfast and dinner, 10 g/time. The serum uric acid level was measured before the intervention and on days 15, 30, 45, 60, 75 and 90 after the intervention; the volume of the joint urate crystals was measured by dual-energy CT before the intervention and 90 days after the intervention to evaluate the dissolution effect of the urate crystals before and after the intervention. Meanwhile, information about symptoms of joint redness and heat, joint swelling, acute attack and joint motility were collected by volunteer self-reporting to evaluate whether the joint mobility is improved or not, and if improved, to evaluate the effect of the intervention on the improvement of the number of gout attacks and joint mobility.

TABLE 16
Serum uric acid data during the intervention period (umol/L, mean + standard deviation)

| | Day 0 (Pre- | | | | | | |
|---|---|---|---|---|---|---|---|
| Group | treatment) | Day 15 | Day 30 | Day 45 | Day 60 | Day 75 | Day 90 |
| Control | 643 ± 67 | 633 ± 73 | 657 ± 64 | 649 ± 58 | 671 ± 69 | 641 ± 55 | 632 ± 76 |
| Composition 1 | 633 ± 58 | 680 ± 69 | 642 ± 65 | 594 ± 61 | 552 ± 53 | 500 ± 67 | 482 ± 57 |
| Composition 2 | 633 ± 58 | 640 ± 69 | 632 ± 65 | 604 ± 61 | 592 ± 53 | 572 ± 67 | 575 ± 57 |
| Composition 3 | 642 ± 54 | 714 ± 73 | 667 ± 42 | 602 ± 82 | 524 ± 65 | 508 ± 78 | 410 ± 71 |
| Composition 4 | 637 ± 49 | 687 ± 54 | 675 ± 71 | 631 ± 43 | 599 ± 65 | 537 ± 31 | 502 ± 78 |
| Composition 5 | 641 ± 45 | 692 ± 61 | 667 ± 87 | 624 ± 56 | 603 ± 31 | 525 ± 67 | 499 ± 67 |
| Composition 6 | 639 ± 59 | 706 ± 66 | 650 ± 53 | 570 ± 53 | 507 ± 61 | 449 ± 67 | 401 ± 67 |
| Composition 7 | 629 ± 42 | 603 ± 54 | 607 ± 42 | 583 ± 51 | 562 ± 24 | 571 ± 62 | 581 ± 83 |

The serum uric acid results during product intervention period (Table 16) showed that after the composition 1, composition 3, composition 4, composition 5, composition 6 and composition 7 interventions, the serum uric acid level of patients with chronic gouty arthritis and tophi increased to different degrees and maintained for about 15 days at least and no more than about 45 days at most, and then decreased and eventually fell below the serum uric acid level before the intervention, and this phenomenon occurred in all of the experiment groups which intervened by containing-probiotics compositions. It is speculated that the reason for this phenomenon is that gout patients have a weaker uric acid excretion capability than healthy individuals, and the probiotics promote the dissolution of urate crystals deposited in the joints, ultimately leading to an increase and then a decrease in serum uric acid level in the early stage of the intervention. The patients with serum uric acid level below 420 μmol/L after interventions with seven different compositions were in a number of 5 (33.33%), 0 (0%), 10 (66.67%), 5 (33.33%), 6 (40.00%), 11 (73.33%), and 1 (6.67%), respectively. Therefore, the intervention effects can be ranked as follows: composition 6 and composition 3>composition 1, composition 4 and composition 5>composition 2>composition 7, and there was no significant difference in the effects of composition 3 and composition 6, and also no significant difference in the effects of composition 1, composition 4 and composition 5. The citrate that is still used clinically in China at present to alkalify urine to further promote uric acid excretion, namely the composition 7, has a certain capability of reducing uric acid in a short term, but has a limited effect of reducing uric acid after long-term use.

### 2. Observation of Oral Compositions for the Effect on the Number of Gout Attacks and Joint Motility

The number of acute gout attacks on the joints of volunteers during the intervention period was collected and statistically analyzed. The results of the self-reported number of gout attacks by the subjects during the intervention period (Table 17) of the intervention groups as compared to that of the control group showed that after the intervention, the number of gout attacks was reduced by all of the compositions, among which composition 3 and composition 6 was the most effective, followed by composition 1, composition 4 and composition 5, and again by composition 2, and composition 7 was the least effective. There was no significant difference in the effects of composition 3 and composition 6, and also no significant difference in the effects of composition 1, composition 4 and composition 5.

TABLE 17
Number of acute gout attacks in volunteers

| Group | Number of attacks |
|---|---|
| Control | 4.76 ± 1.1 |
| Composition 1 | 2.33 ± 1.4 |
| Composition 2 | 3.46 ± 1.2 |
| Composition 3 | 1.63 ± 1.1 |
| Composition 4 | 2.85 ± 0.7 |
| Composition 5 | 2.47 ± 1.2 |
| Composition 6 | 1.54 ± 0.9 |
| Composition 7 | 3.87 ± 1.1 |

Information about symptoms of joint redness and heat and joint swelling self-reported by the volunteers were collected and statistically analyzed. The results are shown in Table 18. The results of the number of volunteers with joint redness and heat and joint swelling of the intervention groups as compared to that of the control group showed that: the composition 1, composition 3, composition 4, composition 5 and composition 6 intervention groups showed increased frequency in the first month, began to decrease from the second month, and decreased significantly in the third month, and there was no significant difference between different composition intervention groups, and also no significant difference between the control group and the composition 2 and composition 7 intervention groups.

The results of the number of volunteers with improved joint motility of the intervention groups as compared to that of the control group showed that: after 90 days of intervention, the joint motility of volunteers in composition 1, composition 3, composition 4, composition 5 and composition 6 intervention groups was significantly improved, wherein the effects of composition 3 and composition 6 were superior to those of composition 4 and composition 5, and the effect of composition 1 was weaker than the effects of four compositions above; there was no significant difference between the control group and the composition 2 and composition 7 intervention groups. Overall, according to the results of the improvement of the joints of volunteers through the evaluation of symptoms of joint redness and heat and joint swelling as well as joint motility, the effects can be ranked as follows: composition 3 and composition 6>composition 4 and composition 5>composition 1>composition 7.

TABLE 18
Improvement of joint motility

| | | | | Number of people with |
|---|---|---|---|---|
| | | | | improved joint motility |
| | | | | after intervention |
| | Joint redness and heat and joint swelling (number of | | | (number of people with |
| | people with symptoms/number of people in the group) | | | improvements/number |
| Groups | Days 0-30 | Days 31-60 | Days 61-90 | of people in the group) |
| Control | 6/15 | 5/15 | 7/15 | 2/15 |
| Composition 1 | 9/15 | 5/15 | 4/15 | 6/15 |
| Composition 2 | 7/15 | 6/15 | 5/15 | 3/15 |
| Composition 3 | 12/15 | 3/15 | 2/15 | 12/15 |
| Composition 4 | 13/15 | 5/15 | 3/15 | 8/15 |
| Composition 5 | 9/15 | 4/15 | 1/15 | 9/15 |
| Composition 6 | 12/15 | 5/15 | 2/15 | 11/15 |
| Composition 7 | 5/15 | 3/15 | 6/15 | 3/15 |

### 3. Observation of Oral Compositions for the Effect of Dissolving Urate Crystals

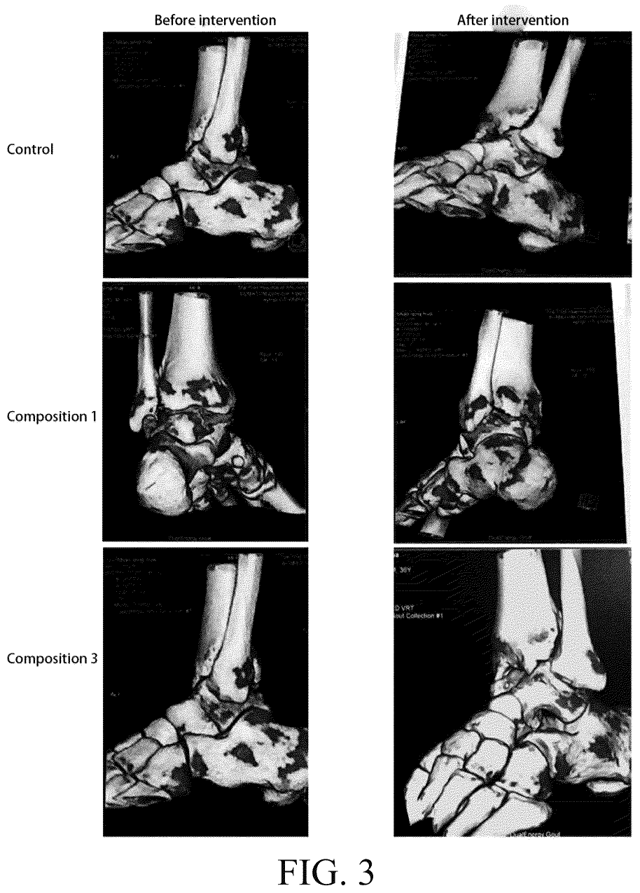

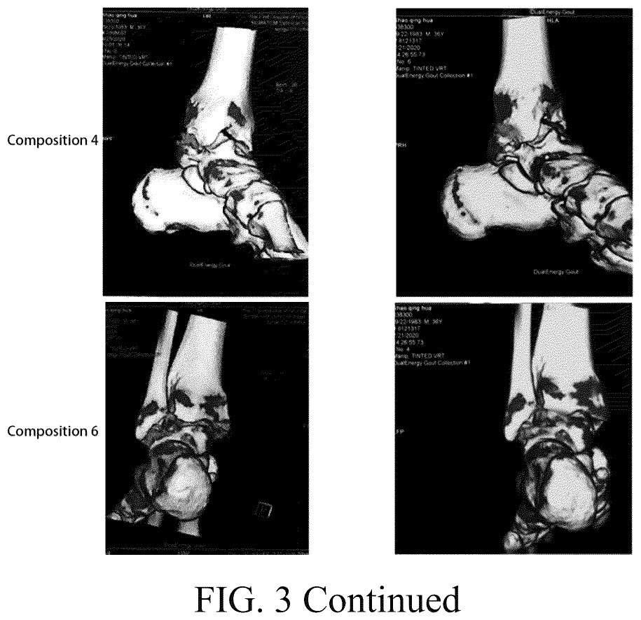

FIG. 3 shows dual-energy CT imaging results of a control group and experiment groups before and after interventions with composition 1, composition 3, composition 4 and composition 6, respectively, and Table 19 shows the change in the volume of urate crystals deposited after the intervention as compared to those before the intervention. It can be seen from the results that urate crystals decreased after the intervention as compared to those before the intervention, and urate crystals deposited at joints significantly decreased especially after the interventions with composition 3 and composition 6 as compared to those in the control group; and there is no significant difference between composition 3 and composition 6 in the effect of dissolving urate crystals. In the colors marked on the dual-energy CT film in FIG. 3, purple indicates deposited calcium salt, and green indicates deposited urate crystals. Those results further confirm that the rise of the serum uric acid level early in the intervention was due to the dissolution of urate crystals deposited at the joints.

TABLE 19
Change in volume of urate crystals deposited after intervention as
compared to those before intervention

| | Before intervention | After intervention | Change values |
|---|---|---|---|
| Patient | (cm³) | (cm³) | (cm³) |
| Control | 2.45 ± 0.56 | 2.67 ± 0.74 | 0.21 ± 0.23 |
| (3 patients) | | | |
| Composition 1 | 2.89 ± 0.67 | 2.32 ± 0.41 | −0.57 ± 0.48 |
| (4 patients) | | | |
| Composition 3 | 3.13 ± 0.45 | 1.97 ± 0.75 | −1.16 ± 0.57 |
| (5 patients) | | | |
| Composition 4 | 2.91 ± 0.58 | 2.04 ± 0.63 | −0.87 ± 0.59 |
| (4 patients) | | | |
| Composition 6 | 3.01 ± 0.34 | 1.69 ± 0.67 | −1.32 ± 0.46 |
| (5 patients) | | | |

### 4. Combined Use of a Composition for Dissolving Urate Crystals and Tophi with a Uric Acid-Reducing Drug

The process of dissolving urate crystals and tophi will result in a temporary increase of the serum uric acid level, which may lead to acute gout inflammation. In order to relieve the increase of the serum uric acid level caused by the dissolution of urate crystals or tophi, the effect of combined use of the composition 6 with a small-dose uric acid-reducing drug on the inhibition of the rise of the serum uric acid level was investigated.

Fifty hyperuricemia patients with tophi were recruited as volunteers, divided into 5 groups, and separately administered with the composition 6 and the uric acid-reducing drug (febuxostat 20 mg/day or benzbromarone 25 mg/day) alone or in combination. The change of serum uric acid during the intervention period was observed.

TABLE 20
Serum uric acid-reducing effect of the combination of a composition
with a uric acid-reducing drug

| | Day 0 (before | | | |
|---|---|---|---|---|
| group | intervention) | Day 30 | Day 60 | Day 90 |
| Composition 6 | 650 ± 72 | 743 ± 75 | 576 ± 69 | 415 ± 58 |
| Composition 6 + | 613 ± 66 | 580 ± 49 | 411 ± 41 | 390 ± 39 |
| febuxostat | | | | |
| Composition 6 + | 622 ± 59 | 550 ± 66 | 431 ± 53 | 370 ± 53 |
| febuxostat | | | | |
| febuxostat | 631 ± 51 | 571 ± 52 | 478 ± 57 | 416 ± 59 |
| benzbromarone | 640 ± 78 | 593 ± 71 | 463 ± 53 | 405 ± 37 |

It can be seen from Table 20 that small doses of febuxostat or benzbromarone can inhibit the increase of the serum uric acid level caused by the dissolution of urate crystals by composition 6. Examples of the present invention have been described above. However, the present invention is not limited thereto. Any modification, equivalent, improvement and the like made without departing from the spirit and principle of the present invention shall fall within the protection scope of the present invention.

1-3. (canceled)

4. A probiotic-containing composition for reducing serum uric acid, comprising at least one of Lactobacillus reuteri strains KLR-1, KLR-13, or KLR 4;
Lactobacillus reuteri strain KLR-3 and KLR-1;
Lactobacillus reuteri strain KLR-3 and KLR-13;
Lactobacillus reuteri strain KLR-3, KLR-1 and KLR-13;
Lactobacillus reuteri strain KLR-3 and KLR-4; or
Lactobacillus reuteri strain KLR-3, KLR-1, KLR-13, and KLR-4, wherein:
the Lactobacillus reuteri strain KLR-1 is a strain with an accession number of CGMCC No. 18699;
the Lactobacillus reuteri strain KLR-3 is a strain with an accession number of CGMCC No. 18700;
the Lactobacillus reuteri strain KLR-13 is a strain with an accession number of CGMCC No. 19329;
the Lactobacillus reuteri strain KLR-4 is a strain with an accession number of CCTCC No. M2020367; and
the Lactobacillus reuteri strains KLR-1, KLR-3, KLR-13, and KLR-4 are lyophilized.

5. The probiotic-containing composition according to claim 4, wherein
the Lactobacillus reuteri strain KLR-1 has a viable count of 1×10⁵−5×10¹¹ CFU/g of the composition;
the Lactobacillus reuteri strain KLR-3 has a viable count of 1×10⁵−5×10¹¹ CFU/g of the composition;
the Lactobacillus reuteri strain KLR-13 has a viable count of 1×10⁵−5×10¹¹ CFU/g of the composition; and
the Lactobacillus reuteri strain KLR-4 has a viable count of 1×10⁵−5×10¹² CFU/g of the composition.

6. The probiotic-containing composition according to claim 4, comprising the Lactobacillus reuteri strain KLR-3 and the Lactobacillus reuteri strain KLR-1; or the Lactobacillus reuteri strain KLR-3 and the Lactobacillus reuteri strain KLR-13.

7. A probiotic-containing composition for reducing serum uric acid, comprising the following components in parts by weight: 1-8 parts of a powder of Lactobacillus reuteri strain KLR-1,1-8 parts of a powder of Lactobacillus reuteri strain KLR-3, and 1-8 parts of a powder of Lactobacillus reuteri strain KLR-13, wherein:
the Lactobacillus reuteri strain KLR-1 is a strain with an accession number of CGMCC No. 18699;
the Lactobacillus reuteri strain KLR-3 is a strain with an accession number of CGMCC No. 18700;
the Lactobacillus reuteri strain KLR-13 is a strain with an accession number of CGMCC No. 19329;
the Lactobacillus reuteri strains KLR-1, KLR-3, and KLR-13 are lyophilized;
the Lactobacillus reuteri strain KLR-1 has a viable count of 1×10⁹−1×10¹² CFU/g of the powder;
the Lactobacillus reuteri strain KLR-3 has a viable count of 1×10⁹−1×10¹² CFU/g of the powder; and
the Lactobacillus reuteri strain KLR-3 has a viable count of 1×10⁹−1×10¹² CFU/g of the powder.

8. Use of the Lactobacillus reuteri strain for reducing serum uric acid according to claim 4 in the preparation of a pharmaceutical or a dietary product for preventing and treating hyperuricemia and/or gout.

9. The use according to claim 8, wherein the pharmaceutical is in a dosage form for oral administration, wherein the dosage form is preferably selected from: a solution, a suspension, an emulsion, a powder, a lozenge, a pill, a syrup, a troche, a tablet, a chewing gum, a concentrated syrup, and a capsule.

10. The use according to claim 7, wherein the dietary product includes an ordinary food product, a health-care food product, or a food product for special medical purpose.

11. A composition capable of reducing serum uric acid and dissolving urate crystals and tophi,
comprising a probiotic-containing composition and prebiotics;
wherein the probiotic-containing composition is one or more of Lactobacillus reuteri strain, Lactobacillus fermentum strain, Bacillus coagulans strain, Lactobacillus rhamnosus strain, Lactobacillus casei strain and Lactobacillus plantarum strain.

12. The composition capable of reducing uric acid and dissolving urate crystals and tophi according to claim 11, wherein
the probiotic-containing composition and the prebiotics are added in a weight ratio of (1-80):(1-80)., preferably (8-32):(40-80).

13. The composition capable of reducing serum uric acid and dissolving urate crystals and tophi according to claim 11, wherein
the probiotic-containing composition has a total viable count of 1×10⁶−6×10¹² CFU/g; preferably, the Lactobacillus reuteri strain, the Lactobacillus fermentum strain, the Bacillus coagulans strain, the Lactobacillus rhamnosus strain, the Lactobacillus casei strain and the Lactobacillus plantarum strain have viable counts of 1.2×10⁹−2.1×10¹² CFU/g, 1.1×10⁹−2.0×10¹² CFU/g, 1.3×10⁸−1.5×10¹¹ CFU/g, 1.3×10⁹−2.1×10¹² CFU/g, 1.0×10⁹−1.4×10¹² CFU/g and 5×10⁹−1.2×10¹² CFU/g, respectively.

14. The composition capable of reducing serum uric acid and dissolving urate crystals and tophi according to claim 11, wherein
the Lactobacillus reuteri strain is one or more of Lactobacillus reuteri strain KLR-1 with an accession number of CGMCC No. 18699, the Lactobacillus reuteri strain KLR-3 with an accession number of CGMCC No. 18700, the Lactobacillus reuteri strain KLR-4 with an accession number of CCTCC No. M2020367 and the Lactobacillus reuteri strain KLR-13 with an accession number of CGMCC No. 19329;
and/or the Lactobacillus rhamnosus strain is Lactobacillus rhamnosus strain KLrh-10 with an accession number of CGMCC No. 19711;
and/or the Lactobacillus casei is Lactobacillus casei strain KLca-10 with an accession number of CGMCC No. 19708;
and/or the Lactobacillus plantarum is Lactobacillus plantarum strain KLpl-3 with an accession number of CCTCC No. M2020366.

15. The composition capable of reducing serum uric acid and dissolving urate crystals and tophi according to claim 11,
further comprising a citrate, wherein the citrate is one or more of citric acid, citric acid esters and citric acid salts;
preferably, the probiotic-containing composition, the prebiotics and the citrate are added in a weight ratio of (1-80):(1-80):(1-50), more preferably (8-32):(40-80):(10-30);
further preferably, the citrate is one or more of citric acid, potassium citrate, magnesium citrate, calcium citrate and potassium sodium hydrogen citrate.

16. The composition capable of reducing serum uric acid and dissolving urate crystals and tophi according to claim 11, wherein
the prebiotics are one or more of inulin, fructo-oligosaccharide, galacto-oligosaccharide, xylo-oligosaccharide, raffinose and stachyose.

17. Use of the composition according to claim 11 in the preparation of a dietary product, a pharmaceutical and a health-care product for preventing and treating hyperuricemia and/or gout.

18. Use of the composition according to claim 11 in combination with a uric acid-reducing drug in the preparation of a dietary product, a pharmaceutical and a health-care product for dissolving urate crystals and tophi.

19. The use according to claim 18, wherein the uric acid-reducing drug is one or more of allopurinol, febuxostat, benzbromarone and probenecid.

20. The use according to claim 18, wherein the composition and the uric acid-reducing drug are added in a weight ratio of 100:1-1000:1, preferably 300:1-750:1.
